(19) United States
(12) Patent Application Publication
(10) Pub. No.: US 2018/0010186 A1
(43) Pub. Date: Jan. 11, 2018

(54) Estimating Abdominal Aortic Aneurysm (AAA) Expansion Rate Using Clinical And Genetic Data

(71) Applicant: Mayo Foundation For Medical Education And Research, Rochester, MN (US)

(72) Inventor: Iftikhar J Kullo, Rochester, MN (US)

(21) Appl. No.: 15/586,805

(22) Filed: May 4, 2017

Related U.S. Application Data
(60) Provisional application No. 62/332,189, filed on May 5, 2016.

(51) Int. Cl. C12Q 1/68

(52) U.S. Cl. CPC C12Q 1/6883; C12Q 2600/118; C12Q 2600/156

(57) ABSTRACT

The present invention relates to methods of identifying and using risk factors for estimating aneurysm expansion, including high risk expansion in abdominal aortic aneurysm patients, such factors including associated genetic variants and gender differences. In particular, methods of using genetic risk scores and/or patient genotypes are provided for use, including individualizing surveillance for reducing AAA, reducing growth rates of AAA and reducing rates of aneurysm ruptures.

This invention was made with government support under HG006379 awarded by National Institutes of Health. The government has certain rights in the invention.

### FIELD OF THE INVENTION

The present invention relates to methods of identifying and using risk factors for estimating aneurysm expansion, including high risk expansion in abdominal aortic aneurysm patients, such factors including associated genetic variants and gender differences. In particular, methods of using genetic risk scores and/or patient genotypes are provided for use, including individualizing surveillance for reducing AAA, reducing growth rates of AAA and reducing rates of aneurysm ruptures.

### BACKGROUND

Abdominal Aortic Aneurysm (AAA) is considered a mulifactorial disease with a heritable component. AAA may have transverse diameters of equal or greater than 3.0 cm. Genome-wide association studies (OAS) allowed the identification of several common variants associated with AAA. Prevalence of AAA is 12.8% in men but merely 4.1% in women above 65 years old. However, women are at higher risk of rupturing an aneurysm than men, but the mechanisms underlying this increased risk are unknown.

Rupture of an abdominal aortic aneurysm (AAA) is associated with an 80% mortality rate. Ruptures occur related to size and aneurysm growth rate.

Therefore, more precise indicators of pending expansion of an aneurysm leading to rupture of an AAA are needed for indicating treatment before aneurysm rupture in a patient.

### SUMMARY OF THE INVENTION

The present invention relates to methods of identifying and using risk factors for estimating aneurysm expansion, including high risk expansion in abdominal aortic aneurysm patients, such factors including associated genetic variants and gender differences. In particular, methods of using genetic risk scores and/or patient genotypes are provided for use, including individualizing surveillance for reducing AAA, reducing growth rates of AAA and reducing rates of aneurysm ruptures.

In one embodiment, the present invention provides a method for identifying and treating a high-risk aneurysm in an Abdominal Aortic Aneurysm (AAA) patient, comprising, a) providing, i) a sample of genomic DNA from an Abdominal Aortic Aneurysm (AAA) patient, and ii) a weighted genetic risk score median calculated using a population of patients with AAA; b) testing said DNA for a single nucleotide polymorphism (SNP) in each of four AAA risk alleles, wherein said risk alleles are rs1466535(C), rs7025486(A), rs2383207(T), and rs599839(G); c) assigning a code for each said individual risk allele; d) calculating a weighted genetic risk score for said patient using said codes for each allele; e) determining that said weighted genetic risk score of said patient is greater than said median; and f) treating said aneurysm of said AAA patient. In one embodiment, said code is a 0 for a non-risk allele homozygote, a 1 for a heterozygote and a 2 for a risk allele heterozygote. In one embodiment, said treating comprises surgical repair to prevent rupture of said aneurysm. In one embodiment, a transverse diameter of said AAA is ≧3.0 cm before said surgical repair. In one embodiment, said patient has history of AAA repair. In one embodiment, said testing of step b) comprises sequencing at least a portion of said DNA sample. In one embodiment, said weighted genetic risk score is a resealed weighted genetic risk score.

In one embodiment, the present invention provides a method for determining increased aneurysm expansion risk and treating an Abdominal Aortic Aneurysm (AAA) patient, comprising, a) providing a sample of genomic DNA from an AAA patient; b) testing said DNA for a single nucleotide polymorphism (SNP) in a single risk allele, where said allele is rs7025486; and c) initiating a treatment when at least one SNP A is present in said allele rs7025486. In one embodiment, said patient is a female. In one embodiment, said testing of step b) comprises sequencing at least a portion of said DNA sample. In one embodiment, said treatment is selected from the group consisting of an arterial de-stiffening and a surgical repair. In one embodiment, wherein a second SNP A is present in said allele rs7025486 said treatment is surgical repair. In one embodiment, a transverse diameter of said AAA is ≧3.0 cm before said surgical repair. In one embodiment, said patient has history of AAA repair. In one embodiment, said risk allele is SORT1-rs599839. In one embodiment, said risk allele SORT1-rs599839 has at least one SNP G. In one embodiment, one said risk allele is tested. In one embodiment, two said risk allele are tested. In one embodiment, said risk allele is tested without testing three risk alleles. In one embodiment, said risk alleles are tested without testing four risk alleles. In one embodiment, said risk allele is associated with increased aneurysm expansion risk without testing a baseline size.

### BRIEF DESCRIPTION OF THE DRAWINGS

FIG. 1. Flow chart of ascertainment of AAA cases and controls in the Vascular Disease Biorepository (VDB). (AAA, abdominal aortic aneurysm; ASCVD, atherosclerotic cardiovascular disease.).

FIG. 2 Association of genetic risk score (GRS) and covariates with presence of AAA in multivariable logistic regression model.

FIG. 3. Aneurysm growth rate based on baseline aneurysm size.

FIG. 4. Aneurysm growth rate in patients with GRS>median versus those GRS≦median.

FIG. 5. Receiver Operating Characteristics (ROC) of (Conventional Risk Factors—CRFs)+genetic risk score (GRS) as calculated herein.

FIG. 6. Difference in mean aneurysm expansion between women and men per A allele of DAB2IP-rs7025486. Y-axis: mean aneurysm expansion (mm/year); F: female; M: male. The slope of F to M indicates difference in mean aneurysm expansion between women and men. −/−:0 risk allele; +/−: 1 risk allele; +/+: 2 risk alleles. Slope of −/− to +/+ indicates increase in mean aneurysm expansion per risk allele. The vertical red dash line at −/−, +/− and +/+ indicates mean aneurysm expansion corresponding to numbers of risk alleles. Blue bars indicate 95% CI of mean aneurysm expansion in women and men. Blue dash curves indicate 95% CI of the slope.

FIG. 7. Mean difference in aneurysm expansion between women and men estimated by DAB2IP-rs7025486[A] in high PP (≧median) vs. low PP group. Comparisons adjusted for gender, baseline AAA size and MAP.

FIG. 8. Blood Pressure over time in AAA patients; men compared to women.

FIG. 9. This chart demonstrates a rapid expansion trajectory and a highGRS group where each associated with increased risk of aneurysm repair at a younger age.

FIG. 10. This chart demonstrates a Kaplan-Meier curve of quartiles of RS2: risk for expansion to a diameter of 5.5 cm.

FIG. 11. This chart demonstrates a C-statistic increase by genetic variants over clinical risk factors alone.

FIG. 12. This chart demonstrates examples of clinical risk factors associated with faster AAA expansion.

FIG. 13 This chart demonstrates examples of genetic variants associated with faster AAA expansion.

FIG. 14A-F. This chart demonstrates examples of an AAA expansion pattern: FIG. 14A-C: early-accelerated pattern; FIG. 14D-F: late-accelerated pattern.

### DEFINITIONS

To facilitate an understanding of the present invention, a number of terms and phrases are defined below. The use of the article “a” or “an” is intended to include one or more. As used herein, terms defined in the singular are intended to include those terms defined in the plural and vice versa.

As used herein, the term “single nucleotide polymorphism” or “SNP” refers to a single base change in a DNA sequence. SNPs are found in one or both alleles of an organism, such as a human. When present in the genome on both chromosomes, an individual is said to be homozygous for a certain polymorphism. When present on a single chromosome, an individual is said to be heterozygous for a certain polymorphism.

An SNP is assigned a unique identifier usually referred to by accession number with a prefix such as “SNP”, “refSNP” or “rs”. An rs followed by a number may refer to one or more SNP positions on a sequence (i.e. multiple when a SNP located in repeated region). When referring to a genotype for an individual, the SNP is specified with the rs number as well as the nucleotide in the allele (i.e. A/A, A/T, T/T, G/G, G/C or C/C, or abbreviated as the change in nucleotide, for example an [A] as the SNP geneotype representing at least one allele having an SNP A nucleotide). Where, A=Adenine, G=Guanine, C=Cytosine, and T=Thymine.

As used herein, the term “aberration” or “abnormality” or “alteration” in singular or plural context refers to a change or deviation. In reference to nucleic acid, such as an SNP, an alteration refers to a difference(s) or a change(s) between DNA nucleotide sequences, including differences between patients with and without AAA.

The term “control” refers to a reference for a test sample, such as control DNA isolated from patients without a known AAA, and the like.

The terms “sample” and “specimen” in the present specification and claims are used in their broadest sense. These terms are also used interchangeably. A sample may be a blood sample, a tissue sample, and the like.

The term “blood sample” refers to whole blood, obtained directly from a subject or during a procedure. Procedures such as clotting, or filtering, or treating with EDTA or Sodium Citrate, and the like, are then used for providing a sample of genomic DNA, for example, in a white blood cell sample, such as peripheral blood mononuclear cells (PBMC), etc.

The terms “nucleic acid sequence” or “nucleotide sequence” or “polynucleotide sequence” as used herein, refer to an oligonucleotide or polynucleotide, and fragments or portions thereof, and to DNA or RNA of genomic, cellular, cell free or synthetic origin which may be single- or double-stranded, and represent the sense or antisense strand.

A “variant” of a first nucleotide sequence is defined as a nucleotide sequence that differs from a similar reference sequence or control sequence, e.g., by having one or more deletions, insertions, or substitutions that may be detected using DNA sequencing and/or digital DNA sequence comparison. For example, comparative digital methods may be used to match an entire region or loci or gene or selected fragment of a first DNA sequence to second DNA sequence for detecting a mutation, such as an SNP.

The terms “patient” and “subject” refer to a mammal that may be treated using the methods of the present invention. “Subject” and “patient” are used herein interchangeably, and a subject may be any mammal but is preferably a human.

A “reference subject” as used herein refers to an individual that provides a basis to which another subject can be compared. In some embodiments, the term “reference subject” refers to a subject that was not diagnosed with AAA, such as a “control subject”.

The term “diagnose” or “diagnosis”, as used herein, refers to the determination, recognition, or identification of the nature, cause, or manifestation of a condition based on signs, symptoms, and/or laboratory findings, such as diagnosing or identifying a subject having AAA.

The term “administering” in reference to a treatment refers to giving a treatment systemically or locally to inhibit growth of AAA and/or inhibit rupture of an AAA. The term “co-administer”, as used herein, refers to a therapy of the administration of two or more agents, drugs, and/or compounds together (i.e. at the same time).

The term “therapy,” used interchangeably herein with “treatment” and variants (e.g., “treating,” “administering”), refers to an attempt to prevent or ameliorate a disease (“abnormal condition,” “disorder,” “syndrome,” etc.), such as AAA, or the symptoms thereof, in a patient or a subject. It is not intended that “treating” a disease require curing or eradicating it, such that the treatment may or may not have a complete therapeutic effect. Therapy can be primary treatment, the first treatment after the initial diagnosis, such as surgery, therapeutics, etc. Therapy can also be treatments after the primary treatment, including follow-up surgery, the same or different therapeutics, therapeutics, life style changes, etc.

The term “MAP” or “mean arterial pressure” refers to the average pressure in a patient's arteries during one cardiac cycle.

The term “pulse pressure” or “PP” refers to blood pressure variation, for example, changes in one or more of left ventricular contractility, heart rate, vascular resistance, elasticity, etc.

### DESCRIPTION OF THE INVENTION

The present invention relates to methods of identifying and using risk factors for estimating aneurysm expansion, including high risk expansion in abdominal aortic aneurysm patients, such factors including associated genetic variants and gender differences. In particular, methods of using genetic risk scores and/or patient genotypes are provided for use, including individualizing surveillance for reducing AAA, reducing growth rates of AAA and reducing rates of aneurysm ruptures.

Methods of the present invention may be used for guiding surveillance testing and determining whether treatment should be initiated for reducing AAA, whether by reducing growth rates of AAA and reducing rates of aneurysm ruptures.

I. A Multi-Locus Genetic Risk Score for Abdominal Aortic Aneurysm. (Zi Ye, et al., “Abstract 37: A Multi-Locus Genetic Risk Score for Abdominal Aortic Aneurysm.” Arteriosclerosis, Thrombosis, and Vascular Biology, 35:A37 2015). Ye, et al., May 5, 2015.

Genome-wide association studies (GWAS) reported several common single nucleotide polymorphisms to be associated with Abdominal Aortic Aneurysm (AAA). It was contemplated that identifying biomarkers of AAA would improve disease prediction and enable individualized screening. Additionally, by identifying a genetic risk factor(s) it may allow improved prediction of the presence of AAA beyond currently known conventional risk factors. Further, this would allow determining whether genetic risk factors would allow prediction of aneurysm growth.

Therefore, we investigated whether a) a multi-locus genetic risk score (GRS) based upon SNPs of GWAS may improve disease prediction beyond conventional risk factors and b) whether a GRS score is associated with aneurysm growth. AAA patients in a case-control study were used for this analysis.

The case control study comprised of 1098 patients with AAA (74±8 years, 83% men) and 6538 controls (67±10 years, 58% men) enrolled in the Mayo Vascular Disease Biorepository. AAA was defined as a transverse diameter of abdominal aorta ≧3.0 cm or history of AAA repair. Controls were participants without known AAA, i.e. no ICD-9 codes of aortic aneurysm. Ascertainment of conventional risk factors and co-morbidities was done using electronic phenotype algorithms. Smoking status was included as part of the analysis. Aneurysm growth rates were determined as latest/pre-operation-first diameter/time interval (mm/yr).

Genomic DNA was extracted from whole blood samples drawn at the time of recruitment. Analysis was done in a Mayo clinic core lab using Illumina infinium Human core Exome Array, Ilumina Humana 610 and 660s Quad-v1 (call rates greater than 95%). Candidate SNPs were from the GWAS catalog and references found in PubMed/NCBI.

A Z-test was used to assess whether the risk estimates of SNPs were substantially different from that in the published literature. Candidate SNPs from independent loci (linkage disequilibrium=0) GRS calculation: r_GRS_W=k/Σᵢwᵢ Σ/i wᵢ×ηᵢ

$$( r\_GRS \_W = \frac{k}{{\sum }_{i}^{ } {w}_{i}} {\sum }_{i}^{ } {w}_{i} \times {n}_{i} , )$$

### (K. Ding, et al., “Genotype-Informed Estimation of Risk of Coronary Heart Disease Based on Genome-Wide Association Data Linked to the Electronic Medical Record.” BMC Cardiovasc Disord, 11:66 2011).

Logistic regression analysis showed the presence of AAA as dependent variable. Therefore, several adjustments were done to the analysis. Adjustments included: 1) age and gender; and 2) additional variables for BMI, hypertension, diabetes, dyslipidemia, ASCVD and family history of aortic aneurysm. AUS and net reclassification index were estimated to assess whether GRS can improve disease prediction beyond conventional risks factors. Aneurysm growth rate was used in at least part of the analysis.

We found five single nucleotide polymorphisms (SNPs) previously shown to be associated with AAA at GWAS significance (P≦10) in NHGRI GWAS catalog and PubMed. A GRS for AAA for each individual was calculated from four SNPs (rs2383107, rs7025486, rs599839, rs1466535) that were replicated in our cohort, by summing the number of risk alleles for each SNP weighted by their estimated effect sizes in GWAS catalog or published largest meta-analysis.

Briefly, results showed that GRS was associated with presence of AAA with an odds ratio (OR) 1.06 (95% confidence interval: 1.03-1.08). The association remained significant after adjustment for age, gender, cardiovascular risk factors, and atherosclerotic cardiovascular diseases. An adjusted OR was 1.05 (1.03-1.08). Further adjustment for each SNP did not attenuate association of GRS with presence of AAA (P<0.001). GRS was not associated with family history of aortic aneurysm (P=0.4).

Adding GRS to conventional risk factors improved net reclassification index by 16% (P<0.001). In a subset of patients with AAA who had sequential imaging studies (n=628), GRS was associated with AAA growth rate ≧1.75 mm/year (median of the cohort) after adjustment for baseline AAA size: adjusted OR: 1.07 (1.00-1.14). In this study, conventional risk factors were not associated with AAA growth. Patients with GRS>5.24 (median of the cohort) had 1.31 times higher odds of having AAA and 1.64 times higher odds of having AAA growth rate ≧1.75 mm/year (P≦0.005) compared to those with GRS≦5.24 (P≦0.005).

TABLE 1
Genetic susceptibility variants for AAA identified from GWAS catalog and
risk allele frequencies: Associations of SNPs with the presence of AAA.

| | | | | In previous publications | | | In VDB | | |
|---|---|---|---|---|---|---|---|---|---|
| | | | | | OR(95% CI) | | | OR(95% CI) | Z test |
| Locus | Gene | SNPs | Effect allele | MAF | P-value | | MAF | P-value | P-value |
| 19p13.2 | LDLR | rs6511720 | G | 0.901 | 1.32 | (1.20-1.43) [7] | 0.9 | 1.04 (0.89-1.21) | 0.009 |
| Bradley DT. 2013 [7] | | | | | 2 × 10 [symbol] ¹⁰ | | | 0.66 | |
| 12q13.3 | LRP1 | rs1466535 | C | 0.58 | 1.15 | (1.10-1.21) [6] | 0.65 | 1.04 (0.94-1.15) | 0.08 |
| Bown MJ. 2011 [6] | | | | | 5 × 10 [symbol] ¹⁰ | | | 0.4 | |
| 9p33.2 | DAB2IP | rs7025846 | A | 0.23 | 1.21 | (1.14-1.28) [9] | 0.27 | 1.17 (1.05-1.29) | 0.54 |
| Gretarsdotti S. 2010 [9] | | | | | 5 × 10 [symbol] ¹⁰ | | | 0.004 | |
| 9p21 | CDKN2A-2B | rs2383207 | T | 0.49 | 1.27 [9] | | 0.52 | 1.22 (1.11-1.34) | 0.51 |
| Gretarsdottir, S. 2010 [9] | | | | | 2 × 10 [symbol] | | | 0.0001 | |
| 1p13.3 | SORT1 | rs599839 | G | 0.22 | 0.81 | (0.76-0.85) [11] | 0.23 | 0.90 (0.80-1.00) | 0.14 |

| | | |
|---|---|---|
| Jones GT, 2013 [11] | 7.2 × 10 [symbol] ¹⁴ | 0.06 |

[6] (Bown, et al., “Abdominal Aortic Aneurysm Is Associated with a Variant in Low-Density Lipoprotein Receptor-Related Protein 1.” Am J Hum Genet, 89: 619-627 2011a)
[7] (Bradley, et al., “A Variant in Ldlr Is Associated with Abdominal Aortic Aneurysm.” Circ Cardiovasc Genet, 6: 498-504 2013).
[9] (Gretarsdottir, et al., “Genome-Wide Association Study Identifies a Sequence Variant within the Dab2ip Gene Conferring Susceptibility to Abdominal Aortic Aneurysm.” Nat Genet, 42: 692-697 2010).
[11] (Jones, et al., “A Sequence Variant Associated with Sortilin-1 (Sort1) on 1p13.3 Is Independently Associated with Abdominal Aortic Aneurysm.” Hum Mol Genet, 22: 2941-2947 2013).
MAF: risk allele frequency; OR: odds ratio; CI = confidence interval; LDLR = low-density lipoprotein receptor; LRP1 = low-density lipoprotein receptor-related protein 1; DAB2IP = DAB2 interacting protein; CDKN2A-2B = Cyclin-dependent kinase inhibitor 2A-2B; SORT1 = Sortilin 1. [symbol]
indicates data missing or illegible when filed

TABLE 2
Patient characteristics.

| — | AAA (n = 1098) | Non-AAA (n = 6538) |
|---|---|---|
| Age, years | 74 (8) | 67 (11) |
| Men | 915 (83) | 4039 (62) |
| Body mass index, kg/m2 | 29.3 (4.9) | 29.1 (5.6) |
| *Smoking (ever) | 952 (87) | 3800 (58) |
| *Hypertension | 908 (82) | 4203 (64) |
| Type 2 diabetes | 342 (25) | 1819 (22) |
| *Dyslipidemia | 959 (87) | 4931 (75) |
| *ASCVD | 966 (88) | 4552 (70) |
| *Family history of aortic | 178 (16) | 471 (7) |
| aneurysm | | |
| *GRS | 5.34 (2.74) | 4.89 (2.86) |

Values expressed as mean (SD) or number (%).
*P-value <0.05 for comparisons in cases vs. non-cases adjusted for age and gender.
Abbreviations:
AAA = abdominal aortic aneurysm;
ASCVD = atherosclerotic cardiovascular disease;
GRS = genetic risk score.

TABLE 3
Age & gender adjusted odds ratio for AAA.

| Covariates | OR (95% CI) |
|---|---|
| GRS above median | 1.37 (1.20-1.56) |
| FHx of AA | 2.49 (2.04-3.02) |
| ASCVD | 2.74 (2.26-3.34) |
| Type 2 diabetes | 1.00 (0.86-1.17) |
| Dyslipidemia | 2.02 (1.67-2.45) |
| Hypertension | 2.43 (2.06-2.89) |
| Smoker | 3.55 (2.96-4.28) |
| BMI 30 kg/m² | 1.02 (0.89-1.17) |

TABLE 4
Multivariable regression analysis: odds ratio for presence of AAA.

| Covariates | OR (95% CI) |
|---|---|
| Age >65 years | 1.55 (1.35-1.77) |
| Male gender | 2.98 (2.52-3.56) |
| GRS above median | 1.31 (1.14-1.50) |
| FHx of AA | 2.43 (1.96-2.98) |
| ASCVD | 2.09 (1.70-2.59) |
| Type 2 diabetes | 0.78 (0.66-0.92) |
| Dyslipidemia | 1.32 (1.07-1.65) |
| Hypertension | 1.86 (1.54-2.26) |
| Smoker | 3.35 (2.77-4.06) |
| BMI 30 kg/m2 | 0.93 (0.80-1.07) |

Conclusions: A multi-locus GRS was associated with presence of AAA and aneurysm growth, suggesting genetic predisposition to disease initiation and progression.
II. Family history of atherosclerotic vascular disease is associated with the presence of abdominal aortic aneurysm. Ye, at al., Vasc Med. 21(1):41-6. Epub 2015 Nov. 12.

We investigated whether family history (FHx) of atherosclerotic cardiovascular disease (ASCVD) was associated with presence of abdominal aortic aneurysm (AAA). The study cohort comprised of 696 patients with AAA (70±8 years, 84% men) and 2686 controls (68±10 years, 61% men) recruited from noninvasive vascular and stress electrocardiogram (ECG) laboratories at Mayo Clinic. AAA was defined as a transverse diameter of abdominal aorta greater than or equal to 3 cm or history of AAA repair. Controls were not known to have AAA. FHx was defined as having at least one first-degree relative with aortic aneurysm or with onset of ASCVD (coronary, cerebral or peripheral artery disease) before age 65 years. FHx of aortic aneurysm or ASCVD were each associated with presence of AAA after adjustment for age, gender, conventional risk factors and ASCVD: adjusted odds ratios (OR; 95% confidence interval): 2.17 (1.66-2.83, p<0.01) and 1.31 (1.08-1.59, p<0.01), respectively. FHx of ASCVD remained associated with AAA after additional adjustment for FHx of aortic aneurysm: adjusted OR: 1.27 (1.05-1.55, p=0.01). FHx of ASCVD in multiple arterial locations was associated with higher odds of having AAA: the adjusted odds were 1.23 times higher for each additionally affected arterial location reported in the FHx (1.08-1.40, p=0.01). Our results suggest both unique and shared environmental and genetic factors mediating susceptibility to AAA and ASCVD.

A. Overview of Determining Whether Family History (FHx) of Atherosclerotic Cardiovascular Disease (ASCVD) was Associated with Presence of Abdominal Aortic Aneurysm (AAA).

Abdominal aortic aneurysm (AAA) is a permanent dilatation of abdominal aorta conventionally defined as a transverse diameter great than or equal to 3.0 cm. It is often asymptomatic until rupture, which is associated with a mortality rate as high as 80%. The prevalence of AAA increases with age, and has been reported to be 12.8% in men and 4.1% in women age >65 years (Pande, et al. Abdominal aortic aneurysm: populations at risk and how to screen. J Vasc Interv Radiol 2008; 19(6 Suppl): S2-8.) No pharmacological treatment is available to effectively limit disease progression. Early identification followed by elective aneurysm repair has been shown to reduce aneurysm-related mortality (Guirguis-Blake, et al. Ultrasonography screening for abdominal aortic aneurysms: a systematic evidence review for the U.S. Preventive Services Task Force. Ann Intern Med 2014; 160: 321-329.) Given the significant disease burden and paucity of treatment options to reduce aneurysm formation and growth, identifying individuals at high risk for AAA may allow tailored screening and improve outcomes. Family history (FHx) is a useful tool for risk assessment, serving as a proxy for genetic predisposition as well as shared environmental factors that contribute to disease development (Kullo, et al. A perspective on the New American College of Cardiology/American Heart Association guidelines for cardiovascular risk assessment. Mayo Clin Proc 2014; 89: 1244-1256.) A positive FHx is a risk factor for coronary heart disease (CHD), cerebrovascular disease (CVD) and peripheral artery disease (PAD) (Go, et al. Heart disease and stroke statistics-2014 update: a report from the American Heart Association. Circulation 2014; 129: e28-e292; Khaleghi, et al. Family history as a risk factor for peripheral arterial disease. Am J Cardiol 2014; 114: 928-932; Khaleghi, et al. Family history as a risk factor for carotid artery stenosis. Stroke 2014; 45: 2252-2256; Reid, et al. Effect of an intervention to improve the cardiovascular health of family members of patients with coronary artery disease: a randomized trial. Can Med Assoc J 2014; 186: 23-30.).

AAA is a multifactorial disease with a significant genetic component 8-10 and risk factors that are shared across subtypes of atherosclerotic cardiovascular disease (ASCVD). (Golledge, et al. Abdominal aortic aneurysm: pathogenesis and implications for management. Arterioscler Thromb Vasc Biol 2006; 26: 2605-2613; Nordon, et al. Pathophysiology and epidemiology of abdominal aortic aneurysms. Nat Rev Cardiol 2011; 8: 92-102). Whether FHx of ASCVD is associated with presence of AAA is unknown. We hypothesized that FHx of ASCVD is a risk factor for AAA. To test this hypothesis, we investigated the association of FHx of ASCVD with presence of AAA in a case-control study of patients referred to the Mayo Clinic. A secondary aim of the study was to assess whether FHx of different subtypes of ASCVD and parental vs. sibling history were differentially associated with presence of AAA.

B. Materials and Methods.

Participants were from the Mayo Clinic Vascular Disease Biorepository (VDB) to identify genetic susceptibility, AAA, ASCVD, risk factors genetic susceptibility variants for vascular diseases. The design and selection criteria for VDB have been reported previously (Ye, et al. An electronic medical record-linked biorepository to identify novel biomarkers for atherosclerotic cardiovascular disease. Glob Cardiol Sci Pract 2013; 2013: 82-90.) Briefly, participants included patients who underwent noninvasive vascular evaluation or stress electro-cardiogram (ECO) at the Mayo Clinic. A questionnaire was given to each participant at the time of consent and scanned into the database after completion. Until August 2013, we had recruited 11,814 participants. The biorepository comprises 8062 participants who had given blood samples, including 1493 individuals without AAA, ASCVD, or rare vascular diseases such as vasculitis, fibromuscular dysplasia, etc. Study questionnaires were available in 5146 out of 8062 participants, including 1015 controls. We excluded 203 participants who were adopted by self-report. A total of 696 participants met the criteria for being AAA cases. As controls we included 1671 participants from the vascular disease group who had ASCVD but not AAA, and 1015 without ASCVD or other vascular disease.

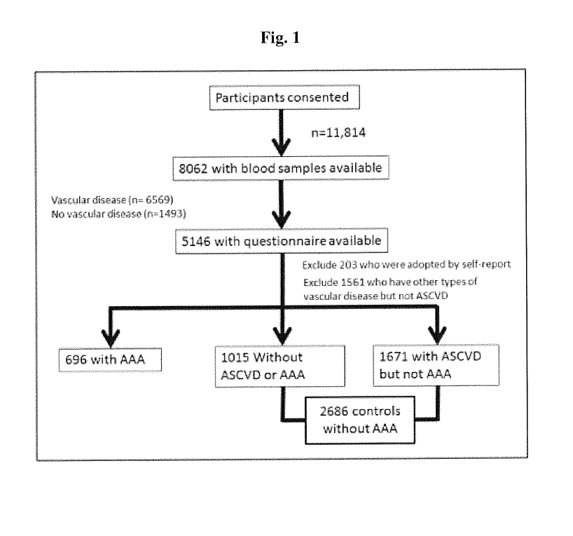

This resulted in a sample of 696 cases and 2686 controls for the analyses (FIG. 1). Participants gave informed consent. The study protocol was approved by the Institutional Review Board of the Mayo Clinic.

We sampled patients based on their AAA status before 31 Dec. 2014. AAA cases were defined as: (1) a distal, infrarenal or juxtarenal abdominal aortic transverse diameter greater than or equal to 3 cm, or (2) history of AAA repair. Controls were patients not known to have AAA. Case status was confirmed by manual review. Controls had no ICD-9 (International Classification of Diseases, Ninth Revision) diagnosis codes for AAA. Prevalent ASCVD, family history and conventional risk factors were ascertained from the study questionnaire. ASCVD was considered present based on physicians' diagnoses of CVD, CHD or PAD, or history of procedures including carotid stenting or endarterectomy, percutaneous coronary intervention or bypass, or revascularization or bypass due to lower extremity arterial stenosis. Hypertension, diabetes and hyperlipidemia were based on self-report (patients were asked if they were ever diagnosed by a physician or were taking antihypertensive, lipid-lowering or hypoglycemic medication), while ever-smoking was defined as a lifetime use of greater than or equal to 100 cigarettes. Patients were asked if their first-degree relatives—father, mother, full sibling, sons and daughters-previously had a myocardial infarction, coronary revascularization or bypass, stroke, carotid endarterectomy, or lower-extremity revascularization or bypass before age 65, and if they had an aortic aneurysm. Details of the questionnaire have been reported previously (Khaleghi, et al. Family history as a risk factor for peripheral arterial disease. Am J Cardiol 2014; 114: 928-932; Khaleghi, et al. Family history as a risk factor for carotid artery stenosis. Stroke 2014; 45: 2252-2256).

Statistics. Descriptive statistics were used to compare demographic and conventional cardiovascular risk factors between cases and controls. Continuous variables were presented as mean (with SD) and dichotomous variables were presented as percentages. Comparisons were performed after adjustment for age and gender. To assess the association of FHx of ASCVD with AAA, logistic regression analysis was performed using the presence of AAA as the dependent variable, first without adjustment and then adjusting for age, gender, body mass index (BMI), hypertension, diabetes, smoking, hyperlipidemia and ASCVD. Analyses were performed stratifying by gender as well.

Additionally, we stratified patients based on (1) FHx of CHD, PAD or CVD and (2) parental and sibling history. We repeated the analyses to (1) compare the association of FHx of subtypes of ASCVD and (2) parental/sibling history with presence of AAA. Interactions between prevalent ASCVD and FHx of ASCVD/aortic aneurysm/CHD/CVD/PAD were assessed and included in the multivariable regression analyses. A two-sided p<0.05 was considered statistically significant. Analyses were performed using the JMP 11.0 (SAS Institute, Cary, N.C., USA) software.

C. Results.

Patient characteristics are shown in Table 5. Hypertension, hyperlipidemia, history of smoking, FHx of aortic aneurysm and FHx of ASCVD were present more often in patients with AAA than in controls after accounting for differences in age and gender. Prevalence of ASCVD was similar between AAA cases and controls, while diabetes was less prevalent in cases than in controls. FHx of aortic aneurysm and ASCVD were each associated with presence of AAA after adjustment for age and gender.

TABLE 5
Patient characteristics.

| | AAA (n = 696) | Controls (n = 2686) | p-value |
|---|---|---|---|
| Age, years | 70 (8) | 68 (10) | <0.01 |
| Men | 583 (84) | 1650 (61) | <0.01 |
| White | 687 (99) | 2643 (98) | 0.99 |
| Body mass index, kg/m² | 29 (5) | 29 (5) | 0.68 |
| Ever-smoker | 488 (71) | 1583 (59) | <0.01 |
| Hypertension | 552 (79) | 1787 (67) | <0.01 |
| Diabetes | 161 (23) | 720 (27) | <0.01 |
| Hyperlipidemia | 578 (83) | 1996 (74) | <0.01 |
| ASCVD | 463 (67) | 1720 (64) | 0.34 |
| PAD | 186 (27) | 807 (27) | 0.18 |
| CHD | 350 (50) | 1082 (40) | 0.07 |
| CVD | 202 (29) | 626 (23) | 0.05 |
| FHx of aortic aneurysm | 119 (17) | 271 (10) | <0.01 |
| FHx of ASCVD | 373 (54) | 1343 (50) | 0.03 |
| Parental history | 189 (27) | 813 (30) | 0.99 |
| Sibling history | 248 (36) | 678 (25) | 0.21 |
| FHx of ASCVD in | | | |
| different arterial | | | |
| locations | | | |
| FHx of CHD | 320 (46) | 1164 (43) | 0.06 |
| FHx of CVD | 126 (18) | 419 (16) | 0.05 |
| FHx of PAD | 51 (7) | 199 (7) | 0.58 |

Values expressed as mean (SD) for age and body mass index, and number (%) for other variables, Comparisons between body mass index, comorbidities, risk factors and family histories were adjusted for age and gender.
AAA, abdominal aortic aneurysm;
ASCVD, atherosclerotic cardiovascular disease;
PAD, peripheral artery disease;
CHD, coronary heart disease;
CVD, cerebrovascular disease;
FHx, family history.

The associations of FHx with aortic aneurysm and ASCVD remained significant after further adjustment for BMI, hypertension, diabetes, smoking, hyperlipidemia, and ASCVD (Table 6). Patients with FHx of ASCVD had a 27% higher likelihood of having AAA after additional adjustment for FHx of aortic aneurysm (adjusted OR, 95% CI: 1.27, 1.05-1.55, p=0.01).

FHx of CHD and CVD were each associated with presence of AAA in models adjusted for BMI, hypertension, type 2 diabetes, smoking, hyperlipidemia, and ASCVD, whereas FHx of PAD was not associated with presence of AAA (Table 6). In addition, FHx of ASCVD in multiple arterial locations was associated with presence of AAA, with a 23% higher likelihood of having AAA for each additionally affected arterial location present in the FHx (Table 6).

Parental and sibling history of aortic aneurysm was associated with presence of AAA after adjustment for age, gender, BMI, hypertension, diabetes, smoking, hyperlipidemia and ASCVD (Table 6). Sibling history of ASCVD was associated with presence of AAA after adjustment for age and gender and additional covariates listed above, while there was no statistically significant association of parental history of ASCVD with AAA (Table 6).

When we categorized participants based on FHx of aortic aneurysm in addition to ASCVD, using patients with FHx of aortic aneurysm as the reference group, patients with FHx of both aortic aneurysm and ASCVD had higher odds of having AAA (OR, 95% CI: 2.00, 1.48-2.70, p<0.01). The association remained significant after adjustment for age and gender and additional covariates (Table 6). Given the gender difference in AAA, we assessed whether gender was a modifier for the association of FHx with AAA. We did not find that gender modified the association of FHx of ASCVD with presence of AAA. We did not find FHx of ASCVD to be additive to risk factors for AAA as assessed by corrected Akaike information criterion from logistic regression models.

TABLE 6
Associations of FHx of ASCVD and aortic aneurysm with presence of AAA.

| | Model 1 | | Model 2 | |
|---|---|---|---|---|
| | OR (95% CI) | p-value | OR (95% CI) | p-value |
| FHx of aortic aneurysm^{a} | 1.97 (1.54-2.50) | <0.01 | 2.17 (1.66-2.83) | <0.01 |
| Parental history | 1.64 (1.22-2.20) | <0.01 | 1.61 (1.16-2.20) | <0.01 |
| Sibling history | 2.81 (1.96-4.02) | <0.01 | 4.55 (2.90-7.29) | <0.01 |
| **FHx of ASCVD | 1.22 (1.02-1.44) | 0.03 | 1.31 (1.08-1.59) | <0.01 |
| Parental history | 1.00 (0.82-1.21) | 0.99 | 0.98 (0.80-1.20) | 0.85 |
| Sibling history | 1.13 (0.93-1.37) | 0.22 | 1.31 (1.08-1.59) | <0.01 |
| **FHx of ASCVD in different arterial locations | | | | |
| FHx of CHD | 1.18 (1.00-1.40) | 0.06 | 1.20 (1.00-1.44) | 0.04 |
| FHx of CVD | 1.25 (1.00-1.57) | 0.05 | 1.36 (1.05-1.76) | 0.02 |
| FHx of PAD | 1.09 (0.78-1.50) | 0.59 | 1.07 (0.75-1.48) | 0.70 |
| Number of arterial locations | 1.24 (1.10-1.40) | <0.01 | 1.23 (1.08-1.40) | 0.01 |
| involved is FHx of ASCVD | | | | |
| Positive FHx of both aortic | 2.16 (1.58-2.95) | <0.01 | 2.49 (1.74-3.56) | <0.01 |
| aneurysm and ASCVD* | | | | |

Model 1 adjusted for age, gender; Model 2 additionally adjusted for BMI, race, hypertension, diabetes, smoking, hyperlipidemia, ASCVD.
^{a}Reference group in the comparison were patients with FHx of aortic aneurysm.
Interaction of FHx of ASCVD/CHD/CVD/PAD with prevalent ASCVD was assessed.
**Interaction term was included when significant at p < 0.05.
FHx, family history;
ASCVD, atherosclerotic cardiovascular disease;
AAA, abdominal aortic aneurysm;
OR, odds ratio;
CI, confidence interval;
CHD, coronary heart disease;
CVD, cerebrovascular disease;
PAD, peripheral artery disease.

D. Discoveries.

We discovered: (1) FHx of ASCVD was associated with presence of AAA independent of conventional cardiovascular risk factors and FHx of aortic aneurysm; (2) sibling history of ASCVD had a stronger association with AAA than parental history; and (3) FHx of ASCVD in multiple arterial locations increased the odds of having AAA. Our results suggest that both unique and shared environmental and genetic factors mediate disease susceptibility to AAA and ASCVD.

A positive FHx was associated with a two-fold risk of having AAA, with ORs of 1.6-2.5 reported in population-based studies (Golledge, et al. Abdominal aortic aneurysm: pathogenesis and implications for management. Arterioscler Thromb Vasc Biol 2006; 26: 2605-2613.). We found an OR of approximately 2.0 for a positive FHx consistent with previous reports. A novel finding of our study is the association of FHx of ASCVD with presence of AAA. Previous studies have shown several biological pathways to be associated with both FHx of ASCVD and presence of AAA, including inflammatory markers such as C-reactive protein, (Powell, et al. Multifactorial inheritance of abdominal aortic aneurysm. Eur J Vasc Surg 1987; 1: 29-31; Rivera, et al. Association of traditional cardiovascular risk factors with coronary plaque sub-types assessed by 64-slice computed tomography angiography in a large cohort of asymptomatic subjects. Atherosclerosis 2009; 206: 451-457; Hamer, et al. The role of conventional and novel mechanisms in explaining increased risk of cardiovascular events in offspring with positive parental history. J Hypertens 2009; 27: 1966-1971; Golledge, et al. Evaluation of the diagnostic and prognostic value of plasma D-dimer for abdominal aortic aneurysm. Eur Heart J 2011; 32: 354-364) interleukin-6 (Juvonen, et al. Elevated circulating levels of inflammatory cytokines in patients with abdominal aortic aneurysm. Arterioscler Thromb Vasc Biol 1997; 17: 2843-2847; Wallinder, et al. Proinflammatory and anti-inflammatory cytokine balance in patients with abdominal aortic aneurysm and the impact of aneurysm size. Vasc Endovascular Surg 2009; 43: 258-261; Lefkou, et al. Increased levels of proinflammatory cytokines in children with family history of coronary artery disease. Clin Cardiol 2010; 33: E6-10; Rao, et al. Genetic contribution to variation in atherothrombotic phenotypes in the Asian Indian population. The Indian Atherosclerosis Research Study. Thromb Haemost 2009; 102: 379-388) and impaired endothelial function (Gaeta, et al. Arterial abnormalities in the offspring of patients with premature myocardial infarction. N Engl J Med 2000; 343: 840-846; Hamburg, et al. Comparison of endothelial function in young men and women with a family history of premature coronary artery disease. Am J Cardiol 2004; 94: 783-785; Sung, et al. Reduced number and impaired function of circulating endothelial progenitor cells in patients with abdominal aortic aneurysm. Int J Cardiol 2013; 168: 1070-1077; Medina, et al. Relationship between endothelial dependent vasodilation and size of abdominal aortic aneurysms. Ann Vasc Surg 2010; 24: 752-757). Recent genome-wide association studies (GWAS) have revealed several genes to be associated with both ASCVD and AAA, including SORT1 at 1p13.3 (mediating triglyceride metabolism), DAB2IP at 9p33.2 (mediating cell apoptosis and survival), CDKN2A-2B at 9p21 (mediating atherosclerotic plaque formation) and LDLR at 19p13.2, 25-28 suggesting pleiotropic effect of these loci on disease development and common susceptibility genes for both traits. We found that a positive FHx of both ASCVD and aortic aneurysm was associated with higher odds of having AAA than FHx of aortic aneurysm alone (Table 6), consistent with shared genetic susceptibility and environmental risk factors between ASCVD and AAA. We found a stronger association of sibling history of ASCVD with presence of AAA than parental history. The association remained significant after further adjustment for numbers of full brothers and full sisters (analyses not shown). A stronger sibling-sibling association with the presence of CHD and stroke than parental-offspring association has been reported previously (Nasir, et al. Coronary artery calcification and family history of premature coronary heart disease: sibling history is more strongly associated than parental history. Circulation 2004; 110: 2150-2156; Choi, et al. Family history and risk for ischemic stroke: sibling history is more strongly correlated with the disease than parental history. J Neurol Sci 2009; 284(1-2): 29-32). We demonstrate for the first time that a similar pattern exists for AAA. Siblings are more likely to have common environmental factors than parent-offspring pairs. Adverse environment in childhood has been reported to affect risk of atherosclerosis (Smith, et al. Adverse socioeconomic conditions in childhood and cause specific adult mortality: prospective observational study. BMJ 1998; 316: 1631-1635) and death due to cardiovascular disease later in adulthood. (Elo, et al. Socioeconomic status across the life course and all-cause and cause-specific mortality in Finland. Soc Sci Med 2014; 119: 198-206). Alternatively, sibling history may be more easily recalled than remote medical history of parents. We found a differential association of FHx of CHD and CVD with presence of AAA versus that of FHx of PAD with AAA. This could be due to the small number of patients with AAA who had a FHx of of PAD. Recent GWAS reported shared genetic susceptibility variants for ASCVD in different arterial locations (Tragante, et al. The impact of susceptibility loci for coronary artery disease on other vascular domains and recurrence risk. Eur Heart J 2013; 34: 2896-2904). Whether ASCVD in a particular arterial bed is differentially associated with AAA is unclear. The Tromsø study found a carotid athero-sclerosis and CHD to be associated with presence of AAA (Johnsen, et al. Atherosclerosis in abdominal aortic aneurysms: a causal event or a process running in parallel? The Tromso study. Arterioscler Thromb Vase Biol 2010; 30: 1263-1268; Johnsen, et al. Carotid atherosclerosis and relation to growth of infrarenal aortic diameter and follow-up diameter: the Tromso Study. Eur J Vasc Endovasc Surg 2013; 45: 135-140).

Our results suggest that FHx of atherosclerosis in different arterial locations is differentially associated with presence of AAA. Further studies are needed to assess shared and unique genetic susceptibility to ASCVD in different locations and AAA.

Our study included a large cohort (i.e. population) of AAA cases and controls with comprehensive assessment of family history by questionnaire. Subjects were referred to Mayo Clinic, a tertiary care center, which may limit the generalization of these results. A majority of the participants were Caucasian (>98%). Owing to the retrospective nature of this study, there may be a recall bias. For example, an ascertainment of family history was based on participant self-report thus a recall bias may be present. Upon comparison, we did find a similar rate of self-reported family history as that in published population-based studies. Additionally, because some of the controls underwent ultrasound screening, we cannot rule out presence of AAA in controls. However, in a random set of controls (n=50) with at least one abdominal imaging study in the electronic health records (EHR), none had AAA identified as described herein. The association of FHx of ASCVD with presence of AAA did not change when we limited the controls to those with an abdominal imaging study in the EHR (n=2221). The response rate to the study questionnaire was 67%. Therefore, we compared risk profiles of responders versus non-responders. We found that, compared to responders, non-responders were younger, with a higher BMI, and more often were hypertensive or diabetic. However, proportions of men and women and patients with hyperlipidemia or ASCVD were similar between the two groups.

E. Conclusions.

Here we report an association of FHx of ASCVD with presence of AAA in a large cohort of AAA cases and controls. We found that: (1) FHx of ASCVD was associated with presence of AAA independent of conventional risk factors and FHx of aortic aneurysm; (2) sibling history of ASCVD had a stronger association with AAA than parental history; (3) FHx of ASCVD in multiple arterial locations was associated with higher odds of having AAA. Our results suggest that FHx of ASCVD is a risk factor for AAA, and that shared environmental and genetic factors mediate disease susceptibility to both AAA and ASCVD. The presence of FHx of ASCVD may identify patients at increased risk of having AAA, and provide insights on genetic risk for disease development for further investigation.

III. A Multi-Locus Genetic Risk Score for Abdominal Aortic Aneurysm. Ye, et al., Atherosclerosis 246:274-279. Available Online 5 Jan. 2016.

The inventors contemplated whether a multi-locus genetic risk score (GRS) was associated with presence and progression of abdominal aortic aneurysm (AAA) in a case—control study.

### A. A MULTI-LOCUS GRS WAS ASSOCIATED WITH PRESENCE OF AAA AND GREATER ANEURYSM EXPANSION

The study comprised of 1124 patients with AAA (74±8 years, 83% men, 52% of them with a maximal AAA size 5 cm) and 6524 non-cases (67±11 years, 58% men) from the Mayo Vascular Disease Biorepository. AAA was defined as infrarenal abdominal aorta diameter ≧3.0 cm or history of AAA repair. Non-cases were participants without known AAA. A GRS was calculated using 4 SNPs associated with AAA at genome-wide significance (P≦10⁻⁸). The GRS was associated with the presence of AAA after adjustment for age, gender, cardiovascular risk factors, atherosclerotic cardiovascular diseases and family history of aortic aneurysm: odds ratio (OR, 95% confidence interval, CI) 1.06 (1.04-1.09, p<0.001). Adding GRS to conventional risk factors improved the association of presence of AAA (net reclassification index 14%, p<0.001). In a subset of patients with AAA who had ≧2 imaging studies (n=651, mean (SE) growth rate 2.47 (0.11) mm/year during a mean time interval of 5.41 years), GRS, baseline size, diabetes and family history were each associated with aneurysm growth rate in Univariate association (p<0.05). The estimated mean aneurysm growth rate was 0.50 mm/year higher in those with GRS>median (5.78) than those with GRS median (p=0.01), after adjustment for baseline size (p<0.001), diabetes (p=0.046) and family history of aortic aneurysm (p=0.02). Thus, a multi-locus GRS was associated with presence of AAA and greater aneurysm expansion.

### B. OVERVIEW OF STUDY

Abdominal aortic aneurysm (AAA) is conventionally defined as a transverse aortic diameter greater than or equal to 3.0 cm [1]. The prevalence of AAA increases with age and is about 12.8% and 4.1% in men and women >65 years old, respectively [2]. Acute rupture is a devastating outcome that is associated with a high mortality of nearly 80% [3]. Early identification through ultrasound screening followed by elective aneurysm repair has been shown to decrease aneurysm-related mortality [4]. Given the significant disease burden and paucity of treatment options, there is a need to identify biomarkers of AAA that may enable individualized screening.

AAA is a multifactorial disease with a heritable component [5]. Genome-wide association studies (GWAS) have found several common single nucleotide polymorphisms (SNPs) to be associated with AAA [6e11]. Whether such variants can improve prediction of presence of AAA beyond conventional risk factors is unknown. The risk of rupture is associated with aneurysm size and growth rate. Genetic factors that relate to aneurysm growth were unknown. A study of participants in the UK small aneurysm trial found that the 9p21 locus which is associated with atherosclerosis and presence of AAA, was not associated with aneurysm expansion [12]. Whether genetic predisposition to AAA expansion is due to the additive effect of multiple susceptibility alleles is unknown. We contemplated that a multi-locus GRS based on SNPs associated with AAA in GWAS may be useful to improve disease prediction beyond conventional risk factors and might be associated with aneurysm growth.

### C. METHODS

1. Study Participants.

The VDB at Mayo Clinic consists of patients referred for noninvasive vascular evaluation in the Gooda Vascular Center and stress electrocardiographic laboratory, and was initiated in 2008. The design and selection criteria have been reported previously [13]. Briefly, the purpose of this registry is to identify novel biomarkers, including genetic susceptibility markers for common and rare vascular diseases. More than 11,814 adults were recruited. Blood samples of participants were drawn at when recruited. High-density genotyping data were available in 8062 (68%) participants. For the purpose of the current study, we included 7648 (9594.7%) patients, including 1124 with AAA as cases and 6524 non-cases who have ASCVD or were referred for cardio-vascular risk assessment but without ASCVD. Demographic information, conventional risk factors and comorbidities were ascertained by previously validated algorithms using ICD-9-CM diagnosis codes, procedure codes, medication use and laboratory data from the institutional electronic health records (EHR). A questionnaire on physical activity, lifestyle and family history was given to each participant at the time of consent and scanned into the database after completion. Participants gave informed consent. The study protocol was approved by the Institutional Re-view Board of the Mayo Clinic.

2. Ascertainment of Cases and Non-Cases of AAA.

We sampled subjects based on their AAA status. AAA cases were defined as having 1) an infrarenal abdominal aortic diameter ≧3 cm, or 2) a history of open or endovascular AAA repair. Patients with AAA often have similar risk profiles as those with atherosclerotic cardiovascular disease (ASCVD) or have ASCVD concomitantly. To test whether a GRS for AAA can differentiate patients with AAA from those who may have ASCVD, participants not known to have AAA (including lack of billing codes for aortic aneurysm) were selected as non-cases. Such non-cases could have ASCVD in different arterial locations. We manually reviewed 100 non-cases with any abdominal imaging study in the EHR. None of them had AAA mentioned in the radiology report. AAA cases were manually reviewed to confirm the maximal aneurysm size (either anteroposterior or transverse diameter). Radiology reports used to screen included abdominal ultrasound, computerized tomography, magnetic resonance imaging and angiography. To assess AAA progression, the latest or the pre-operation measure of AAA size in the EHRs was collected for AAA cases. Based on previous reports that >85% of adults with ectasia of abdominal aorta will progress to a size ≧3.0 cm [14], and that infrarenal aortic diameter ≧2.5 cm was associated with significantly increased risk of cardiovascular events and mortality compared to those with a diameter <2.5 cm [15], we included aortic size ≧2.5 cm as baseline measure if subsequent measure reaches or exceeds 3 cm (centimeter). Growth rate was used to assess aneurysm expansion, defined as (latest/pre-operation minus first diameter)/time interval (mm/year: millimeter/year). Time interval was calculated in years. We required the shortest follow-up time be at least 3 months for analyses of aneurysm growth.

3. Genotyping and Calculation of GRS.

Genomic DNA was extracted from whole blood samples drawn at the recruitment. Genotyping was performed in Mayo Clinic core lab according to standard protocols using Illumina Infinium Human core Exome Array, and Illumina Human 610 and 660W Quad-v1. Sample call rates were each >95%. Four SNPs were previously genotyped for the participants. SNP rs599839 was imputed using the cosmopolitan 1000 Genomes Project reference panel using SHAPEIT2 for phasing and IMPUTE2 software for imputation. The IMPUTE 2 information score for this SNP was 0.94. SNPs followed Hardy-Weinberg equilibrium (p>0.05). We used logistic regression to estimate the effect in our data set of five SNPs from independent loci (linkage disequilibrium=0) that were associated with AAA at a P-value ≦10⁻⁸ (Table 1). To be conservative in the analyses, we used Z-tests to assess whether the risk estimates of SNPs in our dataset were substantially different from that in the published literature. Except for LDLR (rs65117200, P=0.008 for Z-test), risk estimates for four SNPs were not significantly different from that in previous studies (P>0.05). Therefore, we excluded rs65117200 in the calculation for GRS. We assumed an additive genetic model to construct GRS for each individual by summing the number of risk alleles for each of four SNPs weighted by estimated effect sizes in the GWAS catalog or from the largest meta-analysis and then rescaled by the number of SNPs divided by summed effect size of each SNP, as reported previously [16].

4. Ascertainment of Cardiovascular Risk Factors and ASCVD.

Demographic information was abstracted from the EHR as structured data and conventional cardiovascular risk factors (hypertension, diabetes and dyslipidemia) and ASCVD were ascertained by previously validated algorithms using ICD-9 billing codes and natural language processing [17]. Family history of aortic aneurysm in first-degree relatives and smoking status were ascertained from the study questionnaire. Participants were considered smokers if they had smoked more than 100 cigarettes in the past [18,19]. ASCVD was defined as a history of having any of coronary heart disease, stroke, carotid arterial stenosis or peripheral arterial disease.

5. Statistical Methods and Calculations.

Descriptive statistics were used to compare demographic information and conventional cardiovascular risk factors between cases and non-cases. Continuous variables were presented as mean (standard deviation) and dichotomous variables as numbers (percentages). Comparisons were performed after adjustment for age and gender. To assess the association of GRS with AAA, logistic regression analysis was performed 1) without adjustment; 2) with adjustment for age and gender; and 3) additionally adjusting for body-mass index (BMI), hypertension, diabetes, smoking, dyslipidemia, ASCVD and family history. To assess whether GRS can improve disease identification beyond conventional risk factors, the C-statistic, net reclassification index (NRI) and integrated discrimination improvement (IDI) were estimated.

The association of GRS with aneurysm growth rate in a linear regression model violated homoscedasticity assumption when both were used as continuous variables. Therefore, we dichotomized GRS based on the median of 651 cases with at least two size measures at an interval ≧3 months. Logistic regression analysis was performed after adjustment for baseline size and other covariates associated with aneurysm growth rate in the Univariate analysis. The association of age, gender and conventional risk factors with aneurysm expansion and interaction with GRS were also assessed. Two sub-analyses were performed to assess: 1) whether GRS can improve disease identification beyond age, gender and smoking history-main factors considered in initiating screening; and 2) whether GRS was associated with clinically “high-risk aneurysm” expansion defined as either an aneurysm growth rate ≧10 mm/year or with unstable features requiring urgent intervention (i.e. impending rupture or penetrating ulcers). Analyses were performed using the R statistical package (version 2.13) and JMP 11.0 (SAS Institute, Cary, N.C.) software.

a. Exemplary Calculations of GRS As A Resealed Weighted Genetic Risk Score.

The following is an exemplary calculation of the genetic risk score (GRS) as a rescaled weighted genetic risk score (r_GRS_W) for use as a GRS as described herein. In one embodiment, a GRS may be calculated for providing a GRS for a population of individuals. In another embodiment, a GRS is used for calculating a score for an individual patient. Thus

$$r\_GRS \_W = \frac{k}{{\sum }_{i}^{ } {w}_{i}} {\sum }_{i}^{ } {w}_{i} \times {n}_{i} ,$$

is r_GRS_W=k/Σᵢwᵢ Σ/i wᵢ×ηᵢ.

In one embodiment, the weighted score equation was derived based on the assumption that the SNPs of interest have independent effects on the disease and contribute to the log risk of the disease in an additive manner. Lin, et al., 2009. The rescaled version of the genetic score shown above, uses a rescaling factor in order to provide a weighted genetic score more comparable to the unweighted genetic score for a cumulative number of alleles. Lin, et al., 2009. An example of steps to construct the parts of this equation are provided below.

A patient is genotyped, from a blood sample or a tissue sample, for having a particular risk allele SNP. Then each SNP is assigned a code, i.e. ‘0’ for a non-risk allele homozygote, ‘1’ for a risk-allele SNP heterozygote, and ‘2’ for both alleles having the risk-allele SNP, i.e. a risk-allele homozygote. Thus SNPᵢ=0, 1 or 2 according to the number of risk alleles for the specific locus in an individual. When a population is used for providing a genetic risk score, then the SNPᵢ is a sum of the codes for each allele for the entire population. In an example where SNP₁=rs7025486(A), SNP₁ has a value of 2 for a patient having 2 risk alleles for rs7025486(A), etc. When there are 3 individuals in a population, one a non-risk allele homozygote, one a risk-allele SNP heterozygote and one a risk-allele homozygote, then SNP_{rs7025486(A)}=0+1+2=3 for use in the equation. ηᵢ is the number of risk alleles for SNPᵢ, for example, when 4 risk alleles are used, then i=1, 2, 3, and 4, with each of the 4 alleles assigned a separate number.

When combining multiple SNPs, a weighted genetic score calculation is used based upon a weighted w value calculated for each allele, i.e. wᵢ. for SNPᵢ. Thus, wᵢ=the logarithm of odds ratio (OR at a 95% CI) calculated for each allele based upon that allele's estimated effect size obtained from a GWAS catalog or published largest meta-analysis. For examples of an OR for each allele, see Table 1 showing OR values obtained from the GWAS catalog at NHGRI-EBI Catalog of published genome-wide association studies https://www.ebi.ac.uk/gwas/search?query-ABDOMINAL AORTIC ANEURYSM#association. Thus, wᵢ=log(ORᵢ). For a weighted genetic risk score, with allele counts across several SNPs, weighted by the logarithm of odds ratio −w₁−SNP₁+w₂×SNP₂+ . . . wᵢ×SNPᵢ.

Then a rescaling factor is used=k/Σᵢwᵢ, where k is the number of SNPs used (i.e. k=4 for a 4 SNP allele calculation), for a rescaled weighted genetic score, calculated by summing k×(w₁×SNP₁+w₂×SNP₂+ . . . wᵢ×SNPᵢ)/(w₁+w₂+ . . . wᵢ).

Equations and calculations are generally described in: (K. Ding, et al., “Genotype-Informed Estimation of Risk of Coronary Heart Disease Based on Genome-Wide Association Data Linked to the Electronic Medical Record.” BMC Cardiovasc Disord, 11:66 2011); (Lin, et al, “Risk prediction of prevalent diabetes in a Swiss population using a weighted genetic score-the CoLaus Study.” Diabetologia, 52(4):600-608, 2009).

A median, i.e. middle, is determined as the middle number of the numbers when lined up lowest to highest. When there are two middle numbers instead of one, then determine the value half way in between these two numbers, i.e. add the two middle numbers together then divide by two.

b. Exemplary Calculations and Determination of a Median.

The following is an exemplary use of a median related to identifying individual patients with AAA using a GRS median from a GRS score calculated for each individual patient.

The study comprised of 1098 patients with AAA (74±8 years, 83% men) and 6538 controls (67±10 years, 58% men) enrolled in the Mayo Vascular Disease Biorepository. AAA was defined as a transverse diameter of abdominal aorta ≧3.0 cm or history of AAA repair. Controls were participants without known AAA. A GRS for AAA for each individual was calculated from four SNPs (rs2383107, rs7025486, rs599839, rs1466535) that were replicated in our cohort/population, by summing the number of risk alleles for each SNP weighted by their estimated effect sizes in GWAS catalog or published largest meta-analysis.

GRS was associated with presence of AAA: odds ratio (OR) (95% confidence interval): 1.06 (1.03-1.08). The association remained significant after adjustment for age, sex, cardiovascular risk factors, and atherosclerotic cardiovascular diseases: adjusted OR: 1.05 (1.03-1.08). In this example, adjustment for each SNP did not attenuate association of GRS with presence of AAA (each SNP P<0.001). GRS was not associated with family history of aortic aneurysm (P=0.4). Adding GRS to conventional risk factors improved net reclassification index by 16% (P<0.001).

In a subset of patients with AAA who had sequential imaging studies (n=28), GRS was associated with AAA growth rate ≧1.75 mm/year (median of the cohort) after adjustment for baseline AAA size: adjusted OR: 1.07 (1.00-1.14). No conventional risk factors were associated with AAA growth.

Patients with GRS>5.24 (median of the cohort) had 1.31 times higher odds of having AAA (P≦0.005) and 1.64 times higher odds of having AAA growth rate ≧1.75 mm/year compared to those with GRS≦5.24 (P≦0.005).

C. Results.

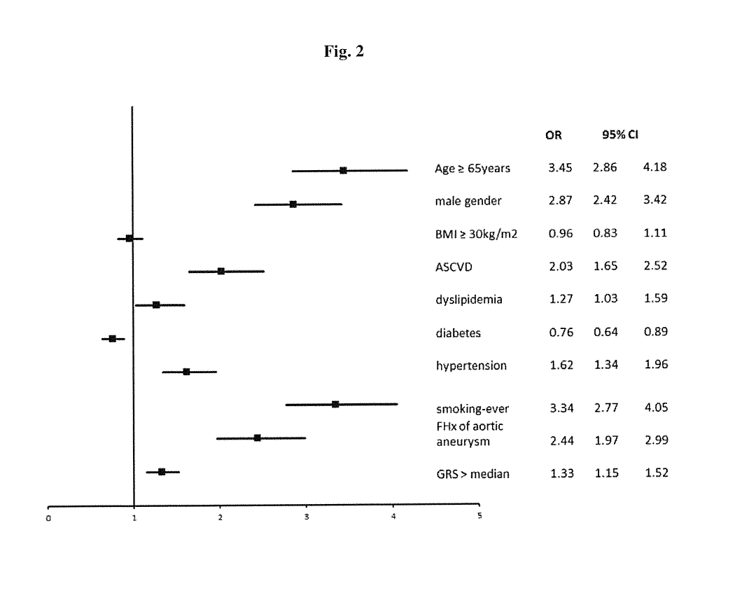

Candidate SNPs associated with AAA and results of z-test are shown in Table 1. We constructed GRS using 4 SNPs after excluding rs6511720 (G). Patient characteristics are shown in Table 7. Patients with AAA had higher prevalence of conventional risk factors and ASCVD than non-cases after adjustment for age and gender, 73 out of 1124 patients with AAA had history of AAA repair. In the remaining patients, the maximal AAA size (mean, SE) was 4.69 (0.04) cm and in 52% AAA size was equal or below 5 cm. The univariate associations of conventional risk factors, ASCVD, family history and GRS with AAA are shown in Table 8. Associations of GRS and covariates with presence of AAA in multivariable logistic regression model are shown in FIG. 2. The GRS was associated with presence of AAA (unadjusted odds ratio, OR, per weighted allele, 95% confidence interval, CI: 1.06, 1.03e1.08, p<0.001). The association remained significant after adjustment for age and gender: adjusted OR (95% CI), 1.06 (1.04e1.08), p<0.001 and further adjustment for body-mass index, hypertension, diabetes, dyslipidemia, smoking, ASCVD and family history: adjusted OR 1.06 (95% CI: 1.04e1.09, p<0.001). We did not find the presence of family history or male gender to alter the association of genetic risk for AAA (P for interaction term P=0.3 for family history*GRS and 0.1 for gender*GRS). Adding GRS to conventional risk factors increased c-statistics from 0.789 (95% CI: 0.776e0.802) to 0.791 (95% CI: 0.777e0.803), a marginal improvement (D=0.002, p=0.049).

TABLE 7
Patient characteristics.

| | AAA | |
|---|---|---|
| | (n = 1124) | Non-AAA (n = 6524) |
| Age, years | 74 (8) | 67 (11) |
| Men | 915 (83) | 4039 (52) |
| Body mass index, kg/m² | 29.3 (4.9) | 29.1 (5.6) |
| *Smoking (ever) | 952 (87) | 3800 (58) |
| *Hypertension | 908 (82) | 4203 (64) |
| Type 2 diabetes | 342 (25) | 1819 (22) |
| *Dyslipidemia | 959 (87) | 4931 (75) |
| *ASCVD | 966 (88) | 4552 (70) |
| *Family history of aortic aneurysm | 178 (16) | 471 (7) |
| *GRS | 5.34 (2.74) | 4.89 (2.86) |

Values expressed as mean (SD) or number (%).
*P-value <0.05 for comparisons in cases vs. non-cases adjusted for age and gender.
Abbreviations:
AAA = abdominal aortic aneurysm;
ASCVD = atherosclerotic cardiovascular disease;
GRS = genetic risk score.

TABLE 8
Univariate association of covariates and GRS with AAA.

| Term | Odds ratio | 95% CI | P-value |
|---|---|---|---|
| Age ≧65 year | 1.68 | 1.48-1.91 | <0.0001 |
| Men | 3.59 | 3.05-4.25 | <0.0001 |
| Body-mass index ≧30 kg/m² | 1.03 | 0.91-1.18 | 0.6 |
| ASCVD | 3.19 | 2.65-3.87 | <0.0001 |
| Dyslipidemia | 2.25 | 1.87-2.72 | <0.0001 |
| Type 2 diabetes | 1.15 | 0.99-1.35 | 0.06 |
| Hypertension | 2.65 | 2.26-3.14 | <0.0001 |
| Smoking (ever) | 2.60 | 2.09-3.28 | <0.0001 |
| Family history of aortic aneurysm | 2.41 | 2.00-2.91 | <0.0001 |
| GRS | 1.06 | 1.03-1.08 | <0.0001 |

Abbreviations:
AAA = abdominal aortic aneurysm;
CI = confidence interval;
ASCVD = atherosclerotic cardiovascular disease;
GRS = genetic risk score.

TABLE 9
Associations of variables with aneurysm growth rate in a multivariable
linear regression model.

| | Regression | | |
|---|---|---|---|
| | coefficient | Std error | P-value |
| Baseline aneurysm size, mm | 1.25 | 0.13 | <0.001 |
| GRS > median | 0.50 | 0.20 | 0.01 |
| Type 2 diabetes | −0.23 | 0.11 | 0.046 |
| Family history of aortic aneurysm | 0.34 | 0.14 | 0.02 |

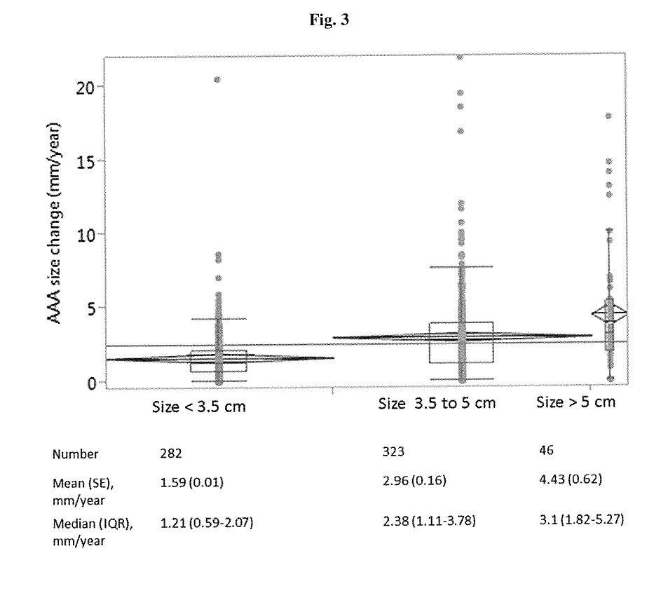

Adding GRS resulted in better disease discrimination manifested by net reclassification index (NRI=0.14, p<0.001). We performed analyses of aneurysm expansion in 651 cases with at ≧2 measures of AAA size (pre-aneurysm repair). We compared patient characteristics in cases included versus those not included in the analyses (Table 10). Briefly, patients included in the analysis were older, more likely to have hypertension, dyslipidemia than those not included, but no difference in mean GRS. The mean (SE) baseline AAA size of 3.69 (0.03) cm and mean (SE) growth rate was 2.47(0.11) mm/year. The mean time interval between two measures was 5.41±3.56 years. The aneurysm growth rate based on baseline size is shown in FIG. 3.

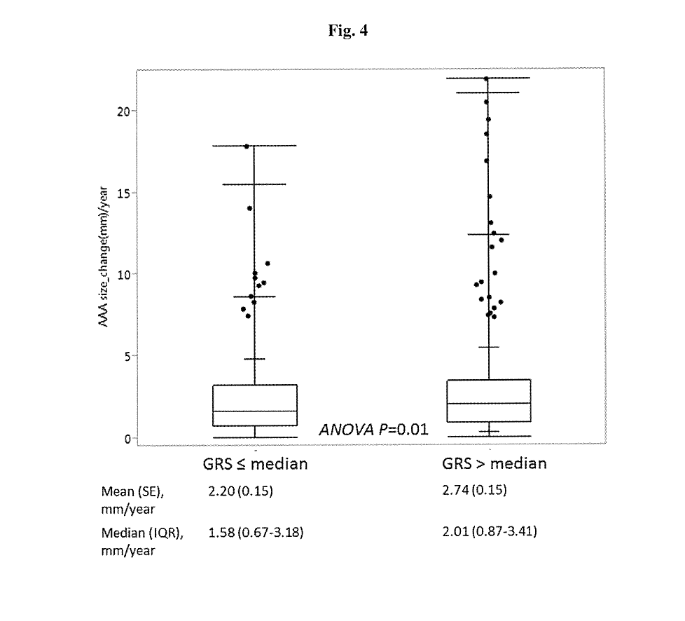

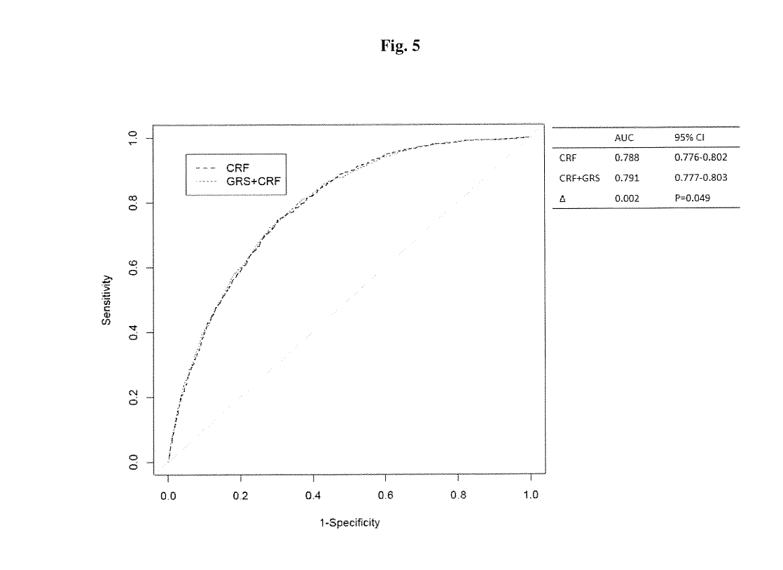

GRS (dichotomized by median), baseline size, diabetes and family history were each associated with aneurysm growth rate in univariate analysis (Table 11). Associations of GRS and covariates with aneurysm growth rate in a multivariable linear regression model are shown in Table 9. The estimated mean aneurysm growth rate was 0.50 mm/year greater in patients with GRS>median than those with GRS median after adjustment for covariates (FIG. 4).

In sub-analysis, adding GRS to a model of age, gender and smoking history improved the c-statistic from 0.770 (95% CI: 0.756e0.784) to 0.773 (95% CI: 0.756e0.786) with significant increase in c-statistics (D=0.003, p=0.02) and improvement in risk discrimination manifested by NRI 14% (p<0.001). 23 out of 651 patients could be classified as having high-risk aneurysm expansion. GRS, BMI and baseline aneurysm size were associated with presence of high-risk aneurysm expansion in univariate association while other covariates were not (Table 12). A higher GRS was associated with 25% greater risk of having high-risk aneurysm expansion (OR, 95% CI: 1.25, 1.06e1.47, p=0.007). The association remained significant after adjustment for BMI and baseline size (adjusted OR, 95% CI: 1.29, 1.08e1.55, p=0.004).

### D. DISCOVERIES

The major findings of our study are: 1) a multi-locus GRS based on 4 susceptibility SNPs was associated with presence of AAA independent of conventional risk factors and family history; 2) a higher GRS was associated with greater aneurysm growth rate independent of baseline abdominal aortic size.

Age, male gender, family history and smoking are major risk factors that are considered when deciding about ultrasound screening for AAA. Such screening has decreased aneurysm-related mortality in men older than 65 years [4,20]. Although women are less likely to have AAA compared to men, women with AAA are at higher risk of aneurysm rupture, higher AAA-related mortality than men [21,22], which may due to delayed detection of the disease. Kent et al. analyzed risk factors for AAA in a population of >3 million, reporting about 50% of the patients with AAA were not eligible for screening based on the current criteria [23]. How to initiate tailored screening and improve disease identification for AAA in a cost-effective manner remains a challenge.

We found that adding GRS to conventional risk factors reclassified 132 patients as cases and 160 as non-cases resulting in a NRI of 14%. In addition, NPV of GRS alone was 0.87, while NPV of age, gender and smoking history was 0.70. These results indicate a potential clinical application of GRS as a screening tool to improve disease detection or to rule out patients with low likelihood of having AAA before initiating imaging studies.

TABLE 10
Patient characteristics: comparison in cases between patients
with AAA progression analysis vs. without progression analysis.

| | Without progression | With progression | |
|---|---|---|---|
| | analysis (n = 473) | analysis (n = 651) | P-value |
| Age, year | 73.2 (8.6) | 74.6 (7.9) | 0.008 |
| Male gender | 395 (84) | 538 (83) | 0.7 |
| BMI, kg/m2 | 29.47 (5.14) | 29.16 (4.74) | 0.3 |
| Hypertension | 374 (79) | 554 (85) | 0.009 |
| Type 2 diabetes | 109 (23) | 167 (26) | 0.3 |
| Dyslipidemia | 384 (81) | 597 (92) | <0.001 |
| Ever smoking | 401 (85) | 572 (88) | 0.1 |
| ASCVD | 408 (86) | 584 (90) | 0.08 |
| Family history of | 82 (17) | 95 (15) | 0.2 |
| aortic aneurysm | | | |
| GRS | 5.33 (2.77) | 5.33 (2.72) | 1.0 |

The risk for aneurysm rupture is mainly determined by size and growth rate. The impact of conventional risk factors on aneurysm growth was debatable. The SMART [26] study reported initial size as the predictor of aneurysm expansion and lack of associations of other risk factors including hypertension, dyslipidemia or ASCVD. A meta-analysis of 18 studies found a higher growth rate in current smokers versus ex/non-smokers [27], whereas the UK Small Aneurysm Trial did not find an association of nicotine level with aneurysm growth [28]. One GWAS [10] reported an association of the 9p21 locus with AAA but not with aneurysm growth.

We found patients with higher GRS (above median) have a higher growth rate after adjustment for baseline size, family history and diabetes, whereas individual SNPs were not associated with aneurysm growth rate after adjustment for baseline size except for DAP2IP (rs7025486) (Table 11). In addition, a higher GRS was associated with higher likelihood of having clinically high-risk aneurysm expansion independent of baseline size. Thus, in some embodiments, DAP2IP (rs7025486[A]) is associated with clinically high-risk aneurysm expansion without measuring a baseline size or incorporating the presence of a baseline size in identifying high-risk aneurysm expansion.

TABLE 11
Univariate associations of variables with aneurysm growth rate.

| | Regression | | |
|---|---|---|---|
| | coefficient | Std error | P-value |
| Age > 65 years | −0.39 | 0.33 | 0.2 |
| Female gender | 0.10 | 0.14 | 0.5 |
| Body-mass index, kg/m² | −0.16 | 0.22 | 0.5 |
| ASCVD | −0.20 | 0.17 | 0.3 |
| Dyslipidemia | 0.16 | 0.19 | 0.4 |
| Type 2 diabetes | −0.32 | 0.12 | 0.01 |
| Hypertension | −0.16 | 0.15 | 0.3 |
| Current smoker | −0.03 | 0.22 | 0.9 |
| Family history of aortic | 0.31 | 0.15 | 0.04 |
| aneurysm | | | |
| GRS > median | 0.53 | 0.21 | 0.01 |
| Baseline aneurysm size, mm | 1.27 | 0.13 | <0.001 |
| DAP2IP (rs7025486) | 0.55 | 0.16 | <0.001 |
| CDKN2A-2B (rs2383207) | 0.19 | 0.15 | 0.2 |
| SORT1 (rs599839) | 0.45 | 0.18 | 0.01 |
| LRP1 (rs1466535) | −0.28 | 0.16 | 0.09 |

DAB2IP is associated with endothelial cell proliferation and survival, regulating cell survival through PI3K-Akt and RAS pathways. Our results indicate that 1) cumulative effects of genetic variants at multiple loci at least partially account for aneurysm expansion; and 2) greater aneurysm growth increases wall stress (stretch) activates DAP2IP protein, accelerating cell apoptosis; or vice versa, a pro-apoptotic effect of DAP2IP may accelerate aneurysm expansion. Regardless, these results indicate that patients with AAA might benefit from tailored monitoring based on the genetic profile.

TABLE 12
Univariate associations of variables
with high-risk aneurysm expansion.

| | OR (95% CI) | p-value |
|---|---|---|
| Age | 1.03 (0.97-1.08) | 0.3 |
| Male gender | 0.75 (0.29-2.30) | 0.6 |
| BMI | 0.87 (0.78-0.96) | 0.007 |
| Hypertension | 1.17 (0.39-5.05) | 0.8 |
| Type 2 diabetes | 0.42 (0.10-1.26) | 0.2 |
| Smoking-ever | 0.64 (0.23-2.27) | 0.4 |
| Dyslipidemia | 0.95-0.27-6.03) | 0.9 |
| ASCVD | 1.21 (0.34-7.68) | 0.8 |
| FHx of aortic aneurysm | 0.87 (0.20-2.61) | 0.8 |
| Baseline aneurysm size | 3.06 (1.84-5.23) | <0.001 |
| GRS-4SNPs | 1.25 (1.06-1.47) | 0.007 |

High-risk aneurysm expansion refers to an aneurysm growth rate ≧1 cm/year or a patient who needs urgent intervention due to rupture or unstable feature of AAA (n=28).

The GRS was associated with dyslipidemia and ASCVD, but not with hypertension, diabetes and smoking (Table 13). No effect modification of dyslipidemia on the associated of GRS with presence of AAA was noted (p for interaction term=0.7). SNPs used to generate the GRS were shown to be associated with lipid traits (LRP1, SORT1) and ASCVD (SORT1, DAB2IP, CDKN2A-2B), indicating pleiotropic effects of these risk variants. However, the association of GRS with AAA remained significant after adjustment for covariates and ASCVD, indicating genetic susceptibility to AAA and both overlapping and unique mechanisms underlying two traits.

Disease-specific GRSs have been reported to improve risk prediction for different cardiovascular diseases [24]. Van't Hof et al. [25] demonstrated that GRSs for lipid traits and coronary heart disease were associated with presence of AAA, suggesting shared genetic background of lipid levels, ASCVD and AAA. To the best of our knowledge, our study is the first to demonstrate that a disease-specific GRS is associated with AAA independent of conventional risk factors and ASCVD. Adding GRS to conventional risk factors marginally increase the c statistic, but improved risk reclassification significantly.

In addition, the OR (1.31, 95% CI 1.14e1.50) of GRS>median for AAA did not significantly change after adjustment for age, gender, BMI, hypertension, type 2 diabetes, smoking, dyslipidemia and ASCVD. Our result suggests that one-time genetic profiling may identify individuals at increased risk for AAA who may benefit from aggressive treatment or life style counseling for modifiable risk factors before genetic and environmental risk factors merge to initiate development of AAA.

TABLE 13
Association of GRS with hypertension or dyslipidemia.

| | Model 1 | Model 2 | Model 3 |
|---|---|---|---|
| | OR | OR | OR |
| | (95% CI) | (95% CI) | (95% CI) |
| | P-value | P-value | P-value |
| Association with | 1.06 | 1.00 | 0.99 |
| hypertension | (0.79-1.41) | (0.99-1.02) | (0.97-1.01) |
| | 0.7 | 0.7 | 0.5 |
| Association with | 1.04 | 1.04 | 1.04 |
| dyslipidemia | (1.02-1.06) | (1.02-1.06) | (1.02-1.06) |
| | <0.001 | <0.001 | <0.001 |

Although GRS was associated with dyslipidemia, we did not find dyslipidemia to modify the association of GRS with presence of AAA (P for interaction term dyslipidemia*GRS=0.7).

### D. BOOTSTRAP ANALYSIS

As additional validation we conducted a bootstrap analysis to assess the effect of adding GRS to a basic model of conventional risk factors in terms of increase in C-statistics (of Conditional Random Field(s) (CRF)), NRI (net reclassification index), and IDI. 1000 bootstrap estimates of these statistics were generated using the same modeling approaches described above and the corresponding 1000 resampled sets of observations. The mean change in AUC (Area Under the Curve), mean NRI, and mean IDI from the 1000 samples as well as 95% CIs defined by the 2.5 and 97.5 percentiles of the 1000 estimates are provided in Table 14. The results confirmed the size, direction, and statistical significance of the values found with the original model.

TABLE 14
Bootstrap validation.

| | | | | P-value for | | | | |
|---|---|---|---|---|---|---|---|---|
| | C-statistics | C-statistics | Increase | increase in | | P-value | | P-value |
| | of CRF | of CRF + GRS | in C-statistics | C-statistics | NRI | for NRI | IDI | for IDI |
| Mean | 0.790 | 0.792 | 0.002 | 0.076 | 0.144 | 0.003 | 0.004 | 0.001 |
| SD | 0.007 | 0.007 | 0.001 | 0.083 | 0.035 | 0.016 | 0.002 | 0.005 |
| Min | 0.765 | 0.766 | 0.000 | 0.000 | 0.032 | 0.000 | 0.001 | 0.000 |
| 2.50% | 0.777 | 0.778 | 0.000 | 0.002 | 0.076 | 0.000 | 0.001 | 0.000 |
| Q1 | 0.785 | 0.787 | 0.001 | 0.020 | 0.120 | 0.000 | 0.003 | 0.000 |
| Median | 0.790 | 0.791 | 0.002 | 0.049 | 0.144 | 0.000 | 0.004 | 0.000 |
| Q3 | 0.794 | 0.796 | 0.002 | 0.100 | 0.168 | 0.000 | 0.005 | 0.000 |
| 97.50% | 0.802 | 0.804 | 0.004 | 0.303 | 0.212 | 0.020 | 0.008 | 0.004 |
| Max | 0.810 | 0.813 | 0.007 | 0.611 | 0.252 | 0.325 | 0.012 | 0.091 |

TABLE 15
Patient characteristics: comparison in controls between
those with imaging study vs. those without imaging study.

| | Without (n = 2856) | With (n = 3668) | P-value |
|---|---|---|---|
| Age, year | 66.2 (10.6) | 68.2 (10.7) | <0.001 |
| Male gender | 1558 (55) | 2236 (61) | <0.001 |
| BMI, kg/m2 | 29.21 (5.74) | 20.08 (5.52) | 0.3 |
| Hypertension | 1504 (53) | 2689 (73) | <0.001 |
| Type 2 diabetes | 527 (18) | 917 (25) | <0.001 |
| Dyslipidemia | 1994 (70) | 2925 (80) | <0.001 |
| Ever smoking | 1561 (55) | 2469 (67) | <0.001 |
| ASCVD | 1786 (63) | 2754 (75) | <0.001 |
| Family history of | 168 (6) | 294 (8) | <0.001 |
| aortic aneurysm | | | |
| GRS | 4.84 (2.84) | 4.94 (2.87) | 0.2 |

Subjects in this study were from a referral population at a tertiary medical center, and the majority was of European ancestry. 60% of cases were included in the sub-analysis of aneurysm growth rate. We compared characteristics in cases included in the progression analysis versus those not included (Table 10). There was no statistically significant difference between two groups. We did not screen the entire control group for AAA. We compared those with abdominal imaging studies and those without (Table 15). Patients without abdominal imaging study were less likely to have risk factors, family history, and ASCVD, but there was no statistical difference in GRS.

Therefore, we used an additive model to build GRS based on the probability that risk for AAA is proportional to the number of risk alleles and that there is no interaction among the loci (we did not find evidence for interaction among SNPs). Genetic variants might have a dominant or recessive effect on AAA, and assuming additivity may be an over-simplification of the true biological mechanism under-lying the disease. However, this is the method employed by the majority studies to assess the association of GRS based on common variants with disease of interest, and to date seems to well approximate the genetic risk for most common diseases.

In conclusion, we demonstrated that a multi-locus GRS was associated with presence of AAA and with aneurysm growth. There is an increasing interest in incorporating findings of common disease risk alleles in the clinical setting [29,30]. Our study suggests the potential of translating results from previous GWAS for AAA for use in the clinical setting, to improve disease identification and risk stratification.

### E. REFERENCES

[1] Go, et al., Heart disease and stroke statisticse 2014 update: a report from the American heart association, Circulation 129 (2014) e28ee292.
[2] Pande, J. A. Beckman, Abdominal aortic aneurysm: populations at risk and how to screen, J. Vasc. Interv. Radiol. 19 (2008) S2e8.
[3] Nordon, R. J. Hinchliffe, I. M. Loftus, M. M. Thompson, Pathophysiology and epidemiology of abdominal aortic aneurysms, Nat. Rev. Cardiol. 8 (2011) 92e102.
[4] Guirguis-Blake, T. L. Beil, C. A. Senger, E. P. Whitlock, Ultrasonography screening for abdominal aortic aneurysms: a systematic evidence review for the U. S. Preventive services task force, Ann. Intern Med. 160 (2014) 321e329.
[5] Powell, R. M. Greenhalgh, Multifactorial inheritance of abdominal aortic aneurysm, Eur. J. Vasc. Surg. 1 (1987) 29e31.
[6] Bown, et al., Abdominal aortic aneurysm is associated with a variant in low-density lipoprotein receptor-related protein 1, Am. J. Hum. Genet. 89 (2011) 619e627.
[7] Bradley, et al., A variant in LDLR is associated with abdominal aortic aneurysm, Circ. Cardiovasc Genet. 6 (2013) 498e504.
[8] Elmore, et al., Identification of a genetic variant associated with abdominal aortic aneurysms on chromosome 3p112.3 by genome wide association, J. Vasc. Surg. 49 (2009) 1525e1531.
[9] Gretarsdottir, et al., Genome-wide association study identifies a sequence variant within the DAB2IP gene conferring susceptibility to abdominal aortic aneurysm, Nat. Genet. 42 (2010) 692e697.
[10] Helgadottir, et al., The same sequence variant on 9p21 associates with myocardial infarction, abdominal aortic aneurysm and intracranial aneurysm, Nat. Genet. 40 (2008) 217e224.
[11] Jones, et al., A sequence variant associated with sortilin-1 (sort1) on 1p13.3 is independently associated with abdominal aortic aneurysm, Hum. Mol. Genet. 22 (2013) 2941e2947.
[12] Tragante, et al., The impact of susceptibility loci for coronary artery disease on other vascular domains and recurrence risk, Eur. Heart J. 34 (2013) 2896e2904.
[13] Ye, F. S. Kalloo, A. K. Dalenberg, I. J. Kullo, An electronic medical record-linked biorepository to identify novel biomarkers for atherosclerotic cardiovascular disease, Glob. Cardiol. Sci. Pract. 2013 (2013) 82e90.
[14] Devaraj, S. R. Dodds, Ultrasound surveillance of ectatic abdominal aortas, Ann. R. Coll. Surg. Engl. 90 (2008) 477e482.
[15] Freiberg, et al., Abdominal aortic aneurysms, increasing infrarenal aortic diameter, and risk of total mortality and incident cardiovascular disease events: 10-year follow-up data from the cardiovascular health study, Circulation 117 (2008) 1010e1017.
[16] Ding, K. R. Bailey, I. J. Kullo, Genotype-informed estimation of risk of coronary heart disease based on genome-wide association data linked to the electronic medical record, BMC Cardiovasc Disord. 11 (2011) 66.
[17] Kullo, et al., Leveraging informatics for genetic studies: use of the electronic medical record to enable a genome-wide association study of peripheral arterial disease, J. Am. Med. Inf. Assoc. 17 (2010) 568e574.
[18] State-specific Secondhand Smoke Exposure and Current Cigarette Smoking Among Adults e United States, 2008, pp. 1232e1235. MMWR Morb Mortal Wkly Rep. 2009; 58.
[19] LeFevre, Screening for abdominal aortic aneurysm: U. S. Preventive services task force recommendation statement, Ann. Intern Med. 161 (2014) 281e290.
[20] Sidloff, et al., Aneurysm global epidemiology study: public health measures can further reduce abdominal aortic aneurysm mortality, Circulation 129 (2014) 747e753.
[21] McPhee, J. S. Hill, M. H. Eslami, The impact of gender on presentation, therapy, and mortality of abdominal aortic aneurysm in the united states, 2001-2004, J. Vasc. Surg. 45 (2007) 891e899.
[22] Mureebe, N. Egorova, J. F. McKinsey, K. C. Kent, Gender trends in the repair of ruptured abdominal aortic aneurysms and outcomes, J. Vasc. Surg. 51 (2010) 9Se13S.
[23] Kent, et al., Analysis of risk factors for abdominal aortic aneurysm in a cohort of more than 3 million individuals, J. Vasc. Surg. 52 (2010) 539e548.
[24] Mega, et al., Genetic risk, coronary heart disease events, and the clinical benefit of statin therapy: an analysis of primary and secondary prevention trials, Lancet 385 (9984) (2015 Jun. 6) 2264e2271.
[25] van't Hof, et al., Impact of inherited genetic variants associated with lipid profile, hypertension, and coronary artery disease on the risk of intracranial and abdominal aortic aneurysms, Circ. Cardiovasc Genet. 6 (2013) 264e270.
[26] Schlosser, et al., Growth predictors and prognosis of small abdominal aortic aneurysms, J. Vasc. Surg. 47 (2008) 1127e1133.
[27] Sweeting, S. G. Thompson, L. C. Brown, J. T. Powell, Meta-analysis of individual patient data to examine factors affecting growth and rupture of small abdominal aortic aneurysms, Br. J. Surg. 99 (2012) 655e665.
[28] Brady, S. G. Thompson, F. G. Fowkes, R. M. Greenhalgh, J. T. Powell, Abdominal aortic aneurysm expansion: risk factors and time intervals for surveillance, Circulation 110 (2004) 16e21.
[29] Kullo, et al., Return of results in the genomic medicine projects of the emerge network, Front. Genet. 5 (2014) 50.
[30] Kullo, G. P. Jarvik, T. A. Manolio, M. S. Williams, D. M. Roden, Leveraging the electronic health record to implement genomic medicine, Genet. Med. 15 (2013) 270e271.
   IV. A DAB2IP Genotype—Gender Interaction is Associated with Abdominal Aortic Aneurysm Expansion.

Rupture of an abdominal aortic aneurysm (AAA) is associated with high mortality. Women are at higher risk of rupture than men, but the mechanisms underlying this increased risk are unknown. We investigated risk factors for aneurysm expansion including genetic variants and gender differences in these associations. The following describes the development of predictive risk factors for aneurysm expansion including genetic variants and gender differences in these associations.

Six hundred fifty (650) patients with AAA [mean age 70-8 years, 17% women] enrolled in the Mayo Clinic Vascular Disease Biorepository, who had ≧2 measures of AAA size, and available high-density genotyping data. We assessed whether variants in 5 susceptibility genes for AAA (CDKN2A-2B, SORT1, DAB2IP, LRP1 and LDLR) were associated with AAA expansion (mm/year) and whether any associations differed by gender.

Results: The mean baseline AAA size was 3.67±0.77 cm. Women had a mean aneurysm expansion 0.41 mm/year greater than men after adjustment for baseline AAA size (p<0.05). In addition to baseline size, mean arterial pressure (MAP), non-diabetic status, SORT-rs599839[G] and DAB2IP-rs7025486[A] were associated with greater aneurysm expansion (each p<0.05).

The associations of MAP and rs599839[G] were similar in both genders (both p-_{interaction}*gender≧0.1); while the associations of baseline size, pulse pressure (PP) and rs7025486[A] were stronger in women than men (p-_{interaction}*gender≦0.02). A three-way interaction of PP*gender*rs7025486[A] was noted in a full-factorial analysis (p=0.007) independent of baseline size and MAP. In the high PP group (≧median), women had a mean growth rate 0.68 mm/year greater per A allele of rs7025486 than men (p-_{interaction}*gender=0.003), whereas there was no difference in the low PP group (p-_{interaction}*gender=0.8).

In conclusion, we demonstrate that DAB2IP-rs7025486[A] and SORT1-rs599839[G] are associated with AAA expansion. The association of rs7025486[A] is stronger in women than men and amplified by high PP, contributing to gender differences in aneurysm expansion.

### A. OVERVIEW OF ABDOMINAL SORTIE ANEURYSM (AAA)

Rupture of abdominal aortic aneurysm (AAA) is associated with a mortality as high as 80% (Norman and Powell, “Abdominal Aortic Aneurysm: The Prognosis in Women Is Worse Than in Men.” Circulation, 115:2865-2869 2007). Larger AAA size, female gender and elevated blood pressure (BP) increase the risk of rupture (Nordon, et al., “Pathophysiology and Epidemiology of Abdominal Aortic Aneurysms.” Nat Rev Cardiol, 8:92-102 2011; Sweeting, et al., “Meta-Analysis of Individual Patient Data to Examine Factors Affecting Growth and Rupture of Small Abdominal Aortic Aneurysms.” Br J Surg, 99:655-665 2012). Baseline AAA size is a determinant of aneurysm expansion. Other risk factors for expansion include higher mean arterial pressure (MAP) or pulse pressure (PP), non-diabetic status and smoking (Bhak et al., “Factors Associated with Small Abdominal Aortic Aneurysm Expansion Rate.” JAMA Surg, 150:44-50 2015; De Rango, et al., “Diabetes and Abdominal Aortic Aneurysms.” Eur J Vasc Endovasc Surg, 47:243-261 2014; Sweeting, et al., “Meta-Analysis of Individual Patient Data to Examine Factors Affecting Growth and Rupture of Small Abdominal Aortic Aneurysms.” Br J Surg, 99:655-665 2012). Aneurysm surveillance to monitor expansion followed by elective AAA repair remains the cornerstone of management (Dua, et al., “Epidemiology of Aortic Aneurysm Repair in the United States from 2000 to 2010.” J Vasc Surg, 59:1512-1517 2014; Guirguis-Blake, et al., “Primary Care Screening for Abdominal Aortic Aneurysm: A Systematic Evidence Review for the U.S. Preventive Services Task Force.” Agency for Healthcare Research and Quality (US), 2014).

AAA is a multifactorial disease with a genetic component (Saratzis and Bown, “The Genetic Basis for Aortic Aneurysmal Disease.” Heart, 100:916-922 2014). Several susceptibility loci in pathways of lipid metabolism (SORT1, LRP1 and LDLR) and cell survival/apoptosis (CDKN2A-2B, DAB21IP) have been reported to be associated with AAA (Bown, et al., “Abdominal Aortic Aneurysm Is Associated with a Variant in Low-Density Lipoprotein Receptor-Related Protein 1.” Am J Hum Genet, 89:619-627 2011 a; Bradley, et al., “A Variant in Ldlr Is Associated with Abdominal Aortic Aneurysm.” Circ Cardiovasc Genet, 6:498-504 2013; Gretarsdottir, et al., “Genome-Wide Association Study Identifies a Sequence Variant within the Dab2ip Gene Conferring Susceptibility to Abdominal Aortic Aneurysm.” Nature genetics, 42:692-697 2010; Helgadottir, et al., “The Same Sequence Variant on 9p21 Associates with Myocardial Infarction, Abdominal Aortic Aneurysm and Intracranial Aneurysm.” Nature genetics, 40:217-224 2008; Jones, et al., “A Sequence Variant Associated with Sortilin-1 (Sort1) on 1p13.3 Is Independently Associated with Abdominal Aortic Aneurysm.” Hum Mol Genet, 22:2941-2947 2013). Whether these variants are associated with aneurysm expansion is unclear. Women have 4 times higher risk of rupture than men and rupture is more likely to occur at a smaller diameter (Bown, et al., “Surveillance Intervals for Small Abdominal Aortic Aneurysms: A Meta-Analysis.” JAMA, 309:806-813 2013; Sweeting, et al., “Meta-Analysis of Individual Patient Data to Examine Factors Affecting Growth and Rupture of Small Abdominal Aortic Aneurysms.” Br J Surg, 99:655-665 2012). Whether risk factors for aneurysm expansion affect women and men differently is not known. Such knowledge will aid in understanding of gender disparity in aneurysm progression and help develop therapies to slow aneurysm expansion.

To this purpose, we studied 650 patients with ≧2 measures of AAA size at least 3 months apart, who had undergone high-density genotyping. We searched National Human Genome Research Institute-European Bioinformatics Institute (NHGRI-EBI) Genome-Wide Association Studies (GWAS) catalog and PubMed for known susceptibility loci for AAA at a genome-wide level significant (p≦5×10⁻⁸). We aimed to assess: 1) whether known genetic susceptibility variants for AAA are associated with aneurysm expansion; and 2) whether these associations differ by gender.

### B. METHODS

1. Study Cohort.

Participants were from the Mayo Vascular Disease Biorepository, an electronic health records (EHR)-linked biorepository of plasma and DNA of patients referred for non-invasive vascular evaluation (Z. Ye, et al., “An Electronic Medical Record-Linked Biorepository to Identify Novel Biomarkers for Atherosclerotic Cardiovascular Disease.” Glob Cardiol Sci Pract, 2013:82-90 2013). The aim of this biorepository is to identify novel biomarkers, including genetic susceptibility variants for common vascular diseases, such as AAA, peripheral artery disease, and carotid artery stenosis, as well as less common vascular diseases such as fibromuscular dysplasia. The biorepository was initiated in 2006 and through August 2014, 11,814 adults had been recruited. High-density genotyping data are available in 8062 (62%) participants. Demographic information, conventional risk factors and comorbidities were ascertained by algorithms based on ICD-9-CM diagnosis codes, procedure codes, medications and laboratory data from EHR. These algorithms have been previously validated in the Electronic Medical Records and Genomics (eMERGE) network (Kullo, et al., “Leveraging Informatics for Genetic Studies: Use of the Electronic Medical Record to Enable a Genome-Wide Association Study of Peripheral Arterial Disease.” J Am Med Inform Assoc, 17:568-574 2010; Z. Ye, et al., “An Electronic Medical Record-Linked Biorepository to Identify Novel Biomarkers for Atherosclerotic Cardiovascular Disease.” Glob Cardiol Sci Pract, 2013:82-90 2013). Participants gave informed consent. The study protocol was approved by the Institutional Review Board of the Mayo Clinic.

2. Ascertainment of AAA And Aneurysm Expansion.

1124 patients with AAA were identified from the Vascular Disease Biorepository. AAA was defined as an infrarenal abdominal aortic diameter ≧3 cm on an imaging study (ultrasound, CT, MRI or angiography reports) or a history of open or endovascular AAA repair. Based on previous reports that >85% of adults with ectasia of abdominal aorta will progress to a size ≧3 cm (Devaraj and Dodds, “Ultrasound Surveillance of Ectatic Abdominal Aortas.” Ann R Coll Surg Engl, 90:477-482 2008) and that infrarenal aortic diameter ≧2.5 cm was associated with significantly increased risk of cardiovascular events and mortality compared to those with a diameter <2.5 cm (Freiberg, et al., “Abdominal Aortic Aneurysms, Increasing Infrarenal Aortic Diameter, and Risk of Total Mortality and Incident Cardiovascular Disease Events: 10-Year Follow-up Data from the Cardiovascular Health Study.” Circulation, 117:1010-1017 2008), we included an aortic size ≧2.5 cm as the baseline measure if the subsequent measure of abdominal aorta exceeded 3 cm. We identified 651 (58%) patients with ≧2 measures of AAA size ≧3 months apart. Aneurysm expansion was estimated as (most recent/pre-repair minus first diameter)/interval (mm/year, follow up until Jan. 24, 2016). We excluded 1 patient with missing BP measures, leaving 650 patients for the analyses.

3. Genotyping.

Genomic DNA was extracted from whole blood samples drawn at recruitment. Genotyping was performed in Mayo Clinic Genotyping Core lab according to standard protocols using Illumina Infimum Human core Exome Array, and Illumina Human 610 and 660W Quad-v1. Sample call rates were >95%. Out of five loci associated with AAA at genome-wide association significance (p≦10⁻⁸) (Table 16), four were previously genotyped and 1 of the single nucleotide polymorphisms (SNP: SORT1-rs599839) was imputed based on the cosmopolitan 1000 Genomes Project reference panel using SHAPEIT2 for phasing (Delaneau, et al., “A Linear Complexity Phasing Method for Thousands of Genomes.” Nat Methods, 9:179-181 2012) and IMPUTE2 software for imputation (Howie, et al., “A Flexible and Accurate Genotype Imputation Method for the Next Generation of Genome-Wide Association Studies.” PLoS Genet, 5:e1000529 2009). The IMPUTE 2 information score for this SNP was 0.94. SNPs were in Hardy-Weinberg equilibrium (each at p>0.05). Risk allele frequencies in our study and those in previous GWAS are listed in Table 1.

4. Ascertainment of Cardiovascular Risk Factors and Atherosclerotic Cardiovascular Disease (ASCVD).

Demographic information was abstracted from the EHR as structured data and conventional cardiovascular risk factors (hypertension, diabetes and dyslipidemia) and ASCVD were ascertained by algorithms validated previously (Kullo, et al., “Leveraging Informatics for Genetic Studies: Use of the Electronic Medical Record to Enable a Genome-Wide Association Study of Peripheral Arterial Disease.” J Am Med Inform Assoc, 17:568-574 2010). Smoking status was ascertained from the study questionnaire.

ASCVD was defined as a history of having any of coronary heart disease, stroke, carotid artery stenosis or peripheral arterial disease. Systolic BP (SBP) and diastolic BP (DBP) measures closest to the baseline and most recent or pre-repair measure of AAA size were manually abstracted from the EHR.

5. Statistical Methods.

Comparisons between women and men were performed by t-test for continuous variables and chi-square test for dichotomous variables. Linear regression analysis was used to assess: 1) univariate associations of conventional risk factors and genetic susceptibility variants with aneurysm expansion; and 2) whether these associations differ by gender after including an interaction term of gender with each candidate risk factor. Additive models of genetic variants were assumed in the analysis. Candidate risk factors for AAA expansion included: age, gender, body-mass index, baseline aneurysm size, hypertension, diabetes, dyslipidemia, current-smoking status, ASCVD and the 5 genetic susceptibility variants.

Given the effect of PP and MAP on aneurysm expansion and rupture (Bhak, et al., “Factors Associated with Small Abdominal Aortic Aneurysm Expansion Rate.” JAMA Surg, 150:44-50 2015; Sweeting, et al., “Meta-Analysis of Individual Patient Data to Examine Factors Affecting Growth and Rupture of Small Abdominal Aortic Aneurysms.” Br J Surg, 99:655-665 2012), we included PP and MAP (2/3 DBP+1/3PP) as risk factors for aneurysm expansion (the average of baseline and most recent or pre-repair BP variables and baseline BP variables were both used in separate models). Stepwise regression analyses with backward elimination were used to identify variables significantly associated with aneurysm expansion, using the criteria p<0.1 to enter and p<0.05 to retain in the model, starting with candidate variables and interaction terms with gender if it was statistically significant (p<0.05) in the univariate analysis. Multivariable regression models were built to assess associations of variables identified from stepwise approach with aneurysm expansion. Additional analyses were performed to assess impact of BP control over time with aneurysm expansion.

### C. RESULTS

Patient characteristics are shown in Table 16. The majority (98%) of participants were Caucasian. Age and prevalence of hypertension, smoking, dyslipidemia and ASCVD were similar in men and women, whereas the prevalence of diabetes was higher in men. Mean PP was higher in women than men, while mean MAP was similar. The mean time-interval between two imaging studies was 5.42 (0.14) years, and was similar in women and men. The mean growth rate was 2.44 (0.1) mm/year. Women had faster aneurysm expansion than men after adjustment for the baseline aneurysm size. Diabetics had slower expansion than non-diabetics (mean±SE: 2.02±0.15 vs. 2.58±0.13 mm/year, p=0.01); the mean growth rate was 1.18 mm/year greater for 1 cm greater in baseline size and 0.4 mm/year greater per 10 mm Hg increase in MAP (both p<0.001). None of the other conventional risk factors were associated with aneurysm expansion.

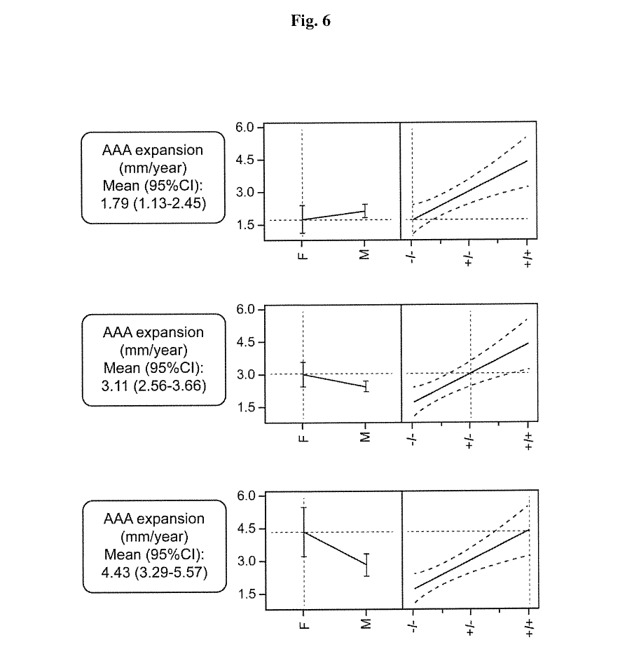

Of 5 genetic susceptibility variants for AAA (Table 17), DAB2IP-rs7025486[A] and SORT1-rs599839[G] were associated with aneurysm expansion: the mean aneurysm expansion was 0.5 mm/year greater per A allele of DAB2IP-rs7025486 (p<0.01) and 0.44 mm/year greater per G allele of SORT1-rs599839 (p<0.01). The association of SORT1-rs599839[G] was similar in women and men. Associations of age, baseline aneurysm size, PP and DAB2IP-rs7025486[A] with aneurysm expansion were different in women and men: older age, higher PP, greater baseline aneurysm size had greater impact in women than men on aneurysm expansion (Table 17). Women had a mean growth rate 0.47 mm/year greater than men per A allele of DAB2IP-rs7025486 (p=0.02). Interactions of gender*0, 1 and 2 A alleles of rs7025486 with aneurysm expansion are shown in FIG. 6. FIG. 6 illustrates: 1) an increase in mean aneurysm expansion corresponds to the numbers of risk alleles; and 2) a greater increase in mean aneurysm expansion in women than men corresponds to the numbers of risk alleles.

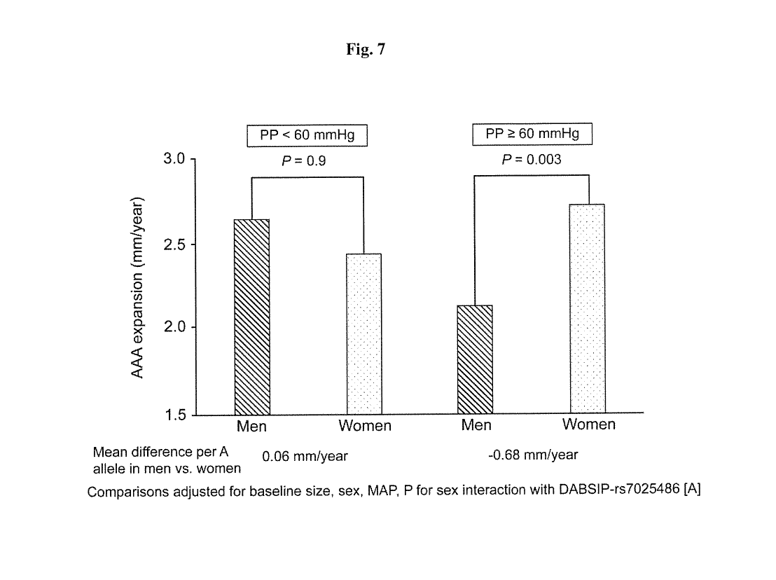

Multivariable stepwise regression analysis identified baseline aneurysm size, MAP, PP, PP*gender, DAB2IP-rs7025486[A], DAB2IP-rs7025486[A]*gender, SORT1 to be independently associated with aneurysm expansion (Table 18): the association of DAB2IP-rs7025486[A] (per risk allele) with aneurysm expansion was stronger in women than men; higher PP was associated with greater aneurysm expansion in women. The mean growth rate was 0.44 mm/year greater in women than men, per A allele of DAB2IP-rs7025486, and 0.30 mm/year greater in women than men for each 10 mm Hg increase in PP, after adjustment for MAP, baseline size and SORT. Given that gender modified the associations of PP and DAB2IP-rs7025486[A] with aneurysm expansion in the same model, we assessed whether PP modified the association of gender*DAB2IP-rs7025486[A] with aneurysm expansion by including a three-way interaction term of PP*gender*DAB2IP-rs7025486[A]. The interaction was significantly associated with greater aneurysm expansion independent of MAP and baseline size (regression coefficient β=0.034, p=0.007, Table 17). Women had a mean growth rate 0.68 mm/year greater than men per A allele of rs7025486 in high PP (≧median) group, but not in low PP group (FIG. 7).

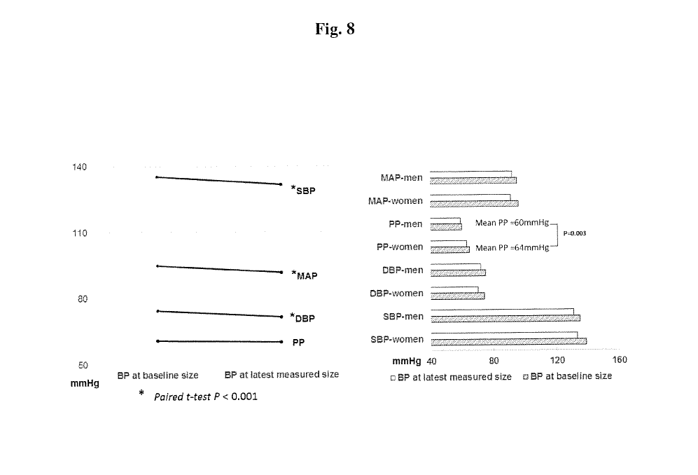

In additional analyses, we found that SBP, DBP and MAP, but not PP decreased over time, (FIG. 8). Change in BP did not modify the association of DAB2IP with aneurysm expansion. When baseline PP and MAP was used in the analysis, results were similar (Table 20).

### D. DISCUSSION

In this study of 650 patients with AAA and with ≧2 measures of AAA size, we confirmed the associations of baseline AAA size, BP measures, and non-diabetic status with aneurysm expansion; in addition, we report for the first time that: a) DAB2IP-rs7025486[A] and SORT1-rs599839[G] are associated with AAA expansion, and b) gender differences in the association of DAB2IP-rs7025486[A] with AAA expansion: the association is stronger in women than men and amplified by higher PP in women.

Prior studies of the genetic basis of AAA expansion included the UK small aneurysm trial (Helgadottir, et al., “The Same Sequence Variant on 9p21 Associates with Myocardial Infarction, Abdominal Aortic Aneurysm and Intracranial Aneurysm.” Nature genetics, 40:217-224 2008) which assessed whether rs10757278[G] at the 9p21 locus was associated with aneurysm expansion in 400 patients with aneurysm diameter 4.5 to 5.5 cm at baseline. The study did not find this locus to be associated with aneurysm growth rate or aneurysm rupture (n=24) during follow-up. In a study of 168 controls and 141 cases of AAA that investigated associations of candidate genes (LRP1, MMP-9, IL-10, AT1R, and MTHFR) with aneurysm expansion (Duellman, et al., “Analysis of Multiple Genetic Polymorphisms in Aggressive-Growing and Slow-Growing Abdominal Aortic Aneurysms.” J Vasc Surg, 60:613-621 e613 2014), borderline significant associations of MMP-9 and LRP1 were noted (p=0.046 and 0.048 respectively). To the best of our knowledge, our study is the first to report associations of genetic variants in SORT1 and DAB2IP with aneurysm expansion and gender-specific genetic susceptibility which is additionally modified by PP.

SORT1 is located at 1p13 locus, coding protein Sortilin, a membrane protein that typically localized to vesicles close to Golgi body, and a sorting molecule that in conjunction with Golgi network, transports lipoproteins and regulates lipoprotein degradation (Dube, et al., “Sortilin: An Unusual Suspect in Cholesterol Metabolism: From Gwas Identification to in Vivo Biochemical Analyses, Sortilin Has Been Identified as a Novel Mediator of Human Lipoprotein Metabolism.” Bioessays, 33:430-437 2011). In animal studies, macrophage SORT) increased/decreased atherosclerosis (Mortensen, et al., “Targeting Sortilin in Immune Cells Reduces Proinflammatory Cytokines and Atherosclerosis.” J Clin Invest, 124:5317-5322 2014; Patel, et al., “Macrophage Sortilin Promotes LDL Uptake, Foam Cell Formation, and Atherosclerosis.” Circ Res, 116:789-796 2015; Strong et al., “Hepatic Sortilin Regulates Both Apolipoprotein B Secretion and LDL Catabolism.” J Clin Invest, 122:2807-2816 2012); while in human studies, over expression of SORT1 decreased LDL cholesterol (Linsel-Nitschke, et al., “Genetic Variation at Chromosome 1p13.3 Affects Sortilin Mrna Expression, Cellular Ldl-Uptake and Serum Ldl Levels Which Translates to the Risk of Coronary Artery Disease.” Atherosclerosis, 208:183-189 2010; Musunuru, et al., “From Noncoding Variant to Phenotype Via Sort1 at the 1p13 Cholesterol Locus.” Nature, 466:714-719 2010). Recent GWAS have identified several susceptibility genetic variants for total/LDL cholesterol (Kathiresan, et al., “Common Variants at 30 Loci Contribute to Polygenic Dyslipidemia.” Nature genetics, 41:56-65 2009; Lettre, et al., “Genome-Wide Association Study of Coronary Heart Disease and Its Risk Factors in 8,090 African Americans: The Nhlbi Care Project.” PLoS Genet, 7:e1001300 2011; Teslovich, et al., “Biological, Clinical and Population Relevance of 95 Loci for Blood Lipids.” Nature, 466:707-713 2010; Willer, et al., “Newly Identified Loci That Influence Lipid Concentrations and Risk of Coronary Artery Disease.” Nature genetics, 40:161-169 2008) and ASCVD (Dichgans, et al., “Shared Genetic Susceptibility to Ischemic Stroke and Coronary Artery Disease: A Genome-Wide Analysis of Common Variants.” Stroke, 45:24-36 2014; Reilly, et al., “Identification of Adamts7 as a Novel Locus for Coronary Atherosclerosis and Association of Abo with Myocardial Infarction in the Presence of Coronary Atherosclerosis: Two Genome-Wide Association Studies.” Lancet, 377:383-392 2011; Schunkert, et al., “Large-Scale Association Analysis Identifies 13 New Susceptibility Loci for Coronary Artery Disease.” Nature genetics, 43:333-338 2011) in non-coding region near gene-3(Dichgans, et al., “Shared Genetic Susceptibility to Ischemic Stroke and Coronary Artery Disease: A Genome-Wide Analysis of Common Variants.” Stroke, 45:24-36 2014; Reilly, et al., “Identification of Adamts7 as a Novel Locus for Coronary Atherosclerosis and Association of Abo with Myocardial Infarction in the Presence of Coronary Atherosclerosis: Two Genome-Wide Association Studies.” Lancet, 377:383-392 2011; Schunkert, et al., “Large-Scale Association Analysis Identifies 13 New Susceptibility Loci for Coronary Artery Disease.” Nature genetics, 43:333-338 2011; Willer, et al., “Newly Identified Loci That Influence Lipid Concentrations and Risk of Coronary Artery Disease.” Nature genetics, 40:161-169 2008) close to SORT or in a non-coding region (Kathiresan. et al., “Common Variants at 30 Loci Contribute to Polygenic Dyslipidemia.” Nature genetics, 41:56-65 2009; Lettre, et al., “Genome-Wide Association Study of Coronary Heart Disease and Its Risk Factors in 8,090 African Americans: The Nhlbi Care Project” PLoS Genet, 7:e1001300 2011; Teslovich, et al., “Biological, Clinical and Population Relevance of 95 Loci for Blood Lipids.” Nature, 466:707-713 2010) that can bind to the enhancer to disrupt Sortilin transcription. SORT1-rs599839[G] has been shown to be associated with increased risk of coronary heart disease/stroke (Dichgans, et al., “Shared Genetic Susceptibility to Ischemic Stroke and Coronary Artery Disease: A Genome-Wide Analysis of Common Variants.” Stroke, 45:24-36 2014) and associated with altered LDL-C levels (Sandhu, et al., “Ldl-Cholesterol Concentrations: A Genome-Wide Association Study.” Lancer, 371:483-491 2008) (Wallace, et al., “Genome-Wide Association Study Identifies Genes for Biomarkers of Cardiovascular Disease: Serum Urate and Dyslipidemia.” Am J Hum Genet, 82:139-149 2008). The association of this genetic variant with AAA expansion and lack of an association of dyslipidemia in our study (regression coefficient±SE: −0.19±0.18, p=0.3) suggests that the association is independent of lipid levels.

DAB2IP encodes DAB interacting protein, also known as apoptosis signal regulating kinase 1 (ASK1)-interacting protein, or AIP1 (anti-inflammatory protein 1), has 14 exons, and is located at 9q33.1-q33.3. The protein is a GTPase-activating protein that regulates cell cycle checkpoint (Xie. et al., “Role of Dab2ip in Modulating Epithelial-to-Mesenchymal Transition and Prostate Cancer Metastasis.” Proc Natl Acad Sci USA, 107:2485-2490 2010), regulates cell growth, mediates TNF-induced cell apoptosis (Ji, et al., “Both Internalization and Aip1 Association Are Required for Tumor Necrosis Factor Receptor 2-Mediated Jnk Signaling.” Arterioscler Thromb Vasc Biol, 32:2271-2279 2012), inhibits JAK-STAT-pathway-dependent vascular smooth cell proliferation (Yu, et al., “Aip1 Prevents Graft Arteriosclerosis by Inhibiting Interferon-Gamma-Dependent Smooth Muscle Cell Proliferation and Intimal Expansion.” Circ Res, 109:418-427 2011) and vascular endothelial growth factor receptor signaling-pathway-dependent endothelial cell migration and angiogenesis (Zhou, et al., “Aip1 Mediates Vascular Endothelial Cell Growth Factor Receptor-3-Dependent Angiogenic and Lymphangiogenic Responses.” Arterioscler Thromb Vasc Biol, 34:603-615 2014). These pathways are associated with extracellular matrix remodeling and inflammation, therefore, could influence aneurysm expansion.

Genetic susceptibility variants in DAB2IP are associated with prostate cancer (Duggan, et al., “Two Genome-Wide Association Studies of Aggressive Prostate Cancer Implicate Putative Prostate Tumor Suppressor Gene Dab2ip.” J Natl Cancer Inst, 99:1836-1844 2007) and ASCVD (Gretarsdottir, et al., “Genome-Wide Association Study Identifies a Sequence Variant within the Dab2ip Gene Conferring Susceptibility to Abdominal Aortic Aneurysm.” Nature genetics, 42:692-697 2010; Harrison, et al., “Association of a Sequence Variant in Dab2ip with Coronary Heart Disease.” Eur Heart J, 33:881-888 2012). In particular, rs7025486[A] is associated with coronary heart disease (Gretarsdottir, et al., “Genome-Wide Association Study Identifies a Sequence Variant within the Dab2ip Gene Conferring Susceptibility to Abdominal Aortic Aneurysm.” Nature genetics, 42:692-697 2010; Harrison et al., “Association of a Sequence Variant in Dab2ip with Coronary Heart Disease.” Eur Heart J, 33:881-888 2012), peripheral artery disease and AAA (Gretarsdottir, et al., “Genome-Wide Association Study Identifies a Sequence Variant within the Dab2ip Gene Conferring Susceptibility to Abdominal Aortic Aneurysm.” Nature genetics, 42:692-697 2010). In contrast to SORT1, LRP1 or LDLR, DAP2IP is not associated with any conventional risk factor, such as hypertension, diabetes or lipids, suggesting that it probably contributes to aneurysm formation and progression independent of the effects of conventional risk factors. Animal studies suggest that estrogen may have protective effect on the integrity of aortic wall through anti-apoptotic (Q. Ding, et al., “Gper-Independent Effects of Estrogen in Rat Aortic Vascular Endothelial Cells.” Mol Cell Endocrinol, 399:60-68 2015), anti-inflammatory effects, inhibition of extracellular matrix remodeling (Laser, et al., “Increased Estrogen Receptor Alpha in Experimental Aortic Aneurysms in Females Compared with Males.” J Surg Res, 186:467-474 2014; Lu, et al., “Dietary Phytoestrogens Inhibit Experimental Aneurysm Formation in Male Mice.” J Surg Res, 188:326-338 2014), promoting cell growth by altering estrogen receptor-DAB2IP pathway (Yeh, et al., “Infiltrating T Cells Promote Renal Cell Carcinoma (Rcc) Progression Via Altering the Estrogen Receptor Beta-Dab2ip Signals.” Oncotarget, 2015). The gender difference in the effect of this variant on aneurysm expansion may be due to lack of protective effect of estrogen in postmenopausal women (Makrygiannis, et al., “Sex Differences in Abdominal Aortic Aneurysm: The Role of Sex Hormones.” Ann Vasc Surg, 28:1946-1958 2014).

An interesting finding is the stronger association of DAB2IP-rs7025486[A] with aneurysm expansion in women in the setting of elevated PP. Higher PP increases shear stress and aortic wall stress, thereby increasing risk for aneurysm expansion (Li, et al., “Association between Aneurysm Shoulder Stress and Abdominal Aortic Aneurysm Expansion: A Longitudinal Follow-up Study.” Circulation, 122:1815-1822 2010), likely mediated through NF-kβ and mitogen-activated protein kinase (MAPK) pathways (Lemarie, et al., “Extracellular Matrix Alterations in Hypertensive Vascular Remodeling.” J Mol Cell Cardiol, 48:433-439 2010)—pathways that underlie cell survival/apoptosis and are regulated by DAB2IP (Zhang, et al., “Aip1-Mediated Stress Signaling in Atherosclerosis and Arteriosclerosis.” Curr Atheroscler Rep, 17:503 2015). Previous studies found significant apoptosis in the stiffened aortic segment located within AAA (Raaz, et al., “Segmental Aortic Stiffening Contributes to Experimental Abdominal Aortic Aneurysm Development.” Circulation, 131:1783-1795 2015) and a greater impact of activation of MAPK pathway on aneurysm expansion in hypertensive female vs. male mice (Schmit, et al., “Hypertension Overrides the Protective Effect of Female Hormones on the Development of Aortic Aneurysm Secondary to Alk5 Deficiency Via Erk Activation.” Am J Physiaol Heart Circ Physiol, 308:H115-125 2015). Aortic wall tension (Eric K. Shang, “Increased Peak Wall Stress in Women with Abdominal Aortic Aneurysms.” Society for Clinical Vascular Surgery 42nd Annual symposium, 2014) and rupture rates of AAA (Sweeting, et al., “Meta-Analysis of Individual Patient Data to Examine Factors Affecting Growth and Rupture of Small Abdominal Aortic Aneurysms.” Br J Surg, 99:655-665 2012) are greater in women than men. PP, a determinant of aortic wall tension that correlates with aneurysm expansion (Guirguis-Blake, et al., “Primary Care Screening for Abdominal Aortic Aneurysm: A Systematic Evidence Review for the U.S. Preventive Services Task Force.” Agency for Healthcare Research and Quality (US), 2014) and rupture (Guirguis-Blake, et al., “Primary Care Screening for Abdominal Aortic Aneurysm: A Systematic Evidence Review for the U.S. Preventive Services Task Force.” Agency for Healthcare Research and Quality (US), 2014; Sweeting, et al., “Meta-Analysis of Individual Patient Data to Examine Factors Affecting Growth and Rupture of Small Abdominal Aortic Aneurysms.” Br J Surg, 99:655-665 2012), is higher in women than men (Mitchell, “Arterial Stiffness and Wave Reflection in Hypertension: Pathophysiologic and Therapeutic Implications.” Curr Hypertens Rep, 6:436-441 2004; Mitchell, et al., “Hemodynamics of Increased Pulse Pressure in Older Women in the Community-Based Age, Gene/Environment Susceptibility-Reykjavik Study.” Hypertension, 51:1123-1128 2008).

The results described herein indicate that wider PP increases the genetic susceptibility to aneurysm expansion in women and may contribute to the increased risk for rupture.

Thus a gender-specific pathogenesis underlying AAA expansion is contemplated in postmenopausal women. Postmenopausal women, without suppression of MAPK pathway mediated by estrogen, may have high PP which in turn increases the genetic susceptibility to AAA expansion through NF-kβ and activation of MAPK pathways that may lead to greater aortic stiffening and increased aortic wall stress in women than men. Subsequent changes at cellular levels lead to aneurysm expansion and rupture.

TABLE 16
Patient Characteristics Stratified By Gender.

| | Women (n = 113) | Men (n = 537) |
|---|---|---|
| Age, years | 70 ± 8 | 70 ± 9 |
| BMI, kg/m² | 28.4 ± 5.5* | 29.3 ± 4.6 |
| Hypertension, % | 89 | 84 |
| Diabetes, % | 18* | 27 |
| Current smoking, % | 39 | 42 |
| Dyslipidemia, % | 91 | 92 |
| ASCVD, % | 87 | 90 |
| PP, mm Hg | 64 ± 15* | 60 ± 13 |
| MAP, mm Hg | 92 ± 11 | 92 ± 10 |
| Baseline AAA size, mm | 35.5 ± 7.2* | 37.2 ± 7.7 |
| Time-interval between 2 imaging | 5.0 ± 3.2 | 5.5 ± 3.7 |
| studies, year | | |
| AAA expansion, mm/year^{†} | 2.9 (0.23)* | 2.6 (0.10) |
| (adjusted for baseline size) | | |

*p < 0.05 for comparisons between women and men by t-test or chi-square test.
^{†}AAA expansion from linear regression model, expressed as least square mean (SE).
PP and MAP: average of BP variables measured at baseline size and most recent or pre-repair size.
BMI: body-mass index;
PP: pulse pressure;
MAP: mean arterial pressure.

TABLE 17
Gender modification of associations of
variables with AAA expansion (mm/year).

| | | | β (SE) for | P for |
|---|---|---|---|---|
| | | | interaction | interaction |
| | | P | term with | term with |
| | β (SE) | value | male gender | male gender |
| Age, years | 0.02 (0.02) | 0.3 | −0.04 (0.02) | 0.01 |
| BMI, kg/m² | −0.01 (0.03) | 0.7 | −0.02 (0.03) | 0.3 |
| Current smoking | −0.15 (0.14) | 0.3 | 0.15 (0.14) | 0.3 |
| Hypertension | −0.07 (0.22) | 0.7 | −0.08 (0.22) | 0.7 |
| Diabetes | −0.33 (0.17) | 0.05 | 0.07 (0.17) | 0.7 |
| Dyslipidemia | −0.03 (0.25) | 1.0 | −0.27 (0.23) | 0.3 |
| ASCVD | −0.16 (0.21) | 0.4 | −0.11 (0.20) | 0.6 |
| PP, mm Hg | 0.01 (0.01) | 0.5 | −0.03 (0.01) | <0.001 |
| MAP, mm Hg | 0.06 (0.01) | <0.001 | −0.02 (0.01) | 0.08 |
| Baseline AAA size, | 1.47 (0.17) | <0.001 | −0.40 (0.17) | 0.02 |
| cm | | | | |
| DAB2IP- | 0.85 (0.21) | <0.001 | −0.47 (0.21) | 0.02 |
| rs7025486[A] | | | | |
| SORT1- | 0.63 (0.22) | 0.005 | −0.69 (0.46) | 0.1 |
| rs599839[G] | | | | |
| CDKN2A-2B- | 0.41 (0.20) | 0.04 | −0.22 (0.43) | 0.3 |
| rs2383207[G] | | | | |
| LRP1- | −0.16 (0.21) | 0.5 | −0.16 (0.21) | 0.4 |
| rs1466535[C] | | | | |
| LDLR- | −0.06 (0.30) | 0.8 | −0.38 (0.30) | 0.2 |
| rs6511720[A] | | | | |

β: regression coefficient; SE: standard error; β of genetic susceptibility variants per risk allele;
PP and MAP: average of BP variables measured at baseline size and most recent or pre-repair size.
BMI: body-mass index;
PP: pulse pressure;
MAP: mean arterial pressure.
MAF: risk allele frequency;
OR: odds ratio;
CI = confidence interval;
LDLR = low density lipoprotein receptor;
LRP1 = low density lipoprotein receptor-related protein 1;
DAB2IP = DAB2 interacting protein;
CDKN2A-2B = Cyclin-dependent kinase inhibitor 2A-2B;
SORT1-Sortilin 1.

TABLE 18
Multivariable regression model of AAA expansion
(mm/year) after stepwise selection

Total (adjusted R² = 0.21)

| | Regression β | |
|---|---|---|
| | (SE) | P-value |
| Baseline size, cm | 1.13 (0.12) | <0.001 |
| Male gender | 1.69 (0.53) | 0.001 |
| PP, mm Hg | −0.01 (0.01) | 0.1 |
| PP*male gender | −0.03 (0.01) | 0.001 |
| MAP, mm Hg | 0.06 (0.01) | <0.001 |
| DAB2IP-rs7025486 (per A allele) | 0.74 (0.19) | <0.001 |
| DAB2IP-rs7025486m*male gender | −0.44 (0.19) | 0.02 |
| SORT1-rs599839 (per G allele) | 0.34 (0.16) | 0.03 |

PP and MAP: average of BP variables measured at baseline size and most recent or pre-repair size.
PP: pulse pressure;
MAP: mean arterial pressure.

TABLE 19
Multivariable linear regression analysis of a
three-way interaction of PP, DAB2IP and gender.

| | Total (adjusted R² = 0.21) | |
|---|---|---|
| | Regression β | |
| | (SE) | P-value |
| Baseline AAA size, cm | 1.13 (0.12) | <0.001 |
| Female gender | −0.44 (0.70) | 0.5 |
| PP, mm Hg | −0.03 (0.01) | 0.005 |
| MAP, mm Hg | 0.06 (0.01) | <0.001 |
| DAB2IP-rs7025486[A] | −1.3 (0.81) | 0.1 |
| SORT1-rs599839[G] | 0.30 (0.15) | 0.05 |
| Female gender*PP | 0.01 (0.01) | 0.6 |
| Female gender*DAB2IP-rs7025486[A] | −1.70 (0.81) | 0.04 |
| DAB2IP-rs7025486[A]*PP | 0.03 (0.01) | 0.01 |
| Female gender*DAB2IP-rs7025486[A]*PP | 0.03 (0.01) | 0.007 |

PP and MAP: average of BP variables measured at baseline size and most recent or pre-repair size.
PP: pulse pressure;
MAP: mean arterial pressure.

TABLE 20
Multivariable regression model after stepwise
selection (baseline PP and MAP).

Total (adjusted R² = 0.16)

| | Regression β | |
|---|---|---|
| | (SE) | P-value |
| Age, year | 0.03 (0.02) | 0.09 |
| Male gender | 6.51 (1.66) | 0.01 |
| Age*male gender | −0.05 (0.02) | 0.003 |
| Body-mass index, kg/m² | /-Not provided | /-Not provided |
| Baseline AAA size, mm | 1.19 (0.13) | <0.001 |
| PP, mm Hg | −0.02 (0.01) | 0.01 |
| MAP, mm Hg | 0.04 (0.01) | <0.001 |
| MAP*male gender | −0.02 (0.01) | 0.01 |
| DAB2IP-rs7025486[A] | 0.92 (0.21) | <0.001 |
| DAB2IP-rs7025486[A]*male | −0.52 (0.21) | 0.01 |
| gender | | |
| SORT1-rs599839[G] | 0.37 (0.17) | 0.03 |

PP: pulse pressure;
MAP: mean arterial pressure.

### E. SUMMARY OF VARIABLES IN THIS STUDY

The majority of participants (98%) were Caucasians referred to a tertiary medical center. Some of the patients had follow-up visits in Mayo Clinic. We compared the characteristics of patients with AAA included in this analysis vs. those not included (n=473). Patients included in the current analysis were older, more likely to be hypertensive and with dyslipidemia than those not included. Prevalence of men, ASCVD, diabetes, smoking history and family history, numbers of risk alleles were similar (analyses not shown). Different from clinical trials, time-intervals between visits during follow-up were not specified for each patient. We did not find current-smoking to be associated with aneurysm expansion in contrast to what was reported in clinical trials (Sweeting, et al., “Meta-Analysis of Individual Patient Data to Examine Factors Affecting Growth and Rupture of Small Abdominal Aortic Aneurysms.” Br J Surg, 99:655-665 2012). This may because the time frame we used to ascertain smoking status was based on the recruitment date and the dates of first and most recent measures of AAA size were not be in this time window.

In conclusion, for the 650 patients with AAA (113 women), in addition to baseline AAA size, BP measures and diabetic status, we found two genetic susceptibility variants for AAA to be associated with aneurysm expansion: DAB2IP-rs7025486[A] and SORT1-rs599839[G]; the association of rs599839[G] is similar in women and men; while the association of rs7025486[A] is stronger in women than men and amplified by higher PP, suggesting that gender modifies genetic susceptibility to aneurysm expansion and this effect is enhanced in the context of higher PP in women.

The timing of surgery, i.e. surgical repair, to prevent aneurysm rupture is associated with AAA size and aneurysm expansion. Risk factors for greater AAA expansion are associated with baseline size, smoking and non-diabetic status. Both genetic susceptibility and environmental factors are implicated in aneurysm formation. Identification of genetic variants in addition to conventional risk factors for aneurysm expansion may lead to individualized management in both men and women. The following are contemplated methods of using genetic information, i.e. genotyping, for initiating treatments.

Translational Outlook 1:

DAB2IP-rs7025486[A] and SORT-rs599839[G] were associated with aneurysm expansion independent of baseline AAA size, suggesting the potential utility of genotyping these variants after AAA detection to optimize surveillance programs to prevent rupture. The stronger association of DAB2IP-rs7025486[A] with aneurysm expansion in women than men suggests the utility of further risk stratification by this SNP in women.

Translational Outlook 2:

The stronger association of DAB2IP-rs7025486[A] with aneurysm expansion in women than men is amplified by higher PP in women, a surrogate of pulsatile load and arterial stiffness, suggesting arterial de-stiffening may have favorable impact in women to limit aneurysm expansion. Nonlimiting examples of de-stiffening therapies are described in Janic, et al., “Review Article: Arterial Stiffness and Cardiovascular Therapy. BioMed Research International, Volume 2014 (2014), Article ID 621437.

The following is an exemplary determination of the probability of aneurysm expansion when at least one allele for rs7025486[A] is present in men and women.

Of 5 genetic susceptibility variants for AAA (Table 17), DAB2IP-rs7025486[A] and SORT1-rs599839[G] were associated with aneurysm expansion. However, the mean aneurysm expansion was 0.5 mm/year greater per A allele of DAB2IP-rs7025486 (p<0.01) over 0.44 mm/year greater per 0 allele of SORT1-rs599839 (p<0.01). While association of SORT)-rs599839[G] was similar in women and men, in women DAB2IP-rs7025486[A] showed a mean growth rate 0.47 mm/year greater than men per A allele of DAB2IP-rs7025486 (p=0.02). FIG. 6 illustrates: 1) an increase in mean aneurysm expansion that corresponds to the numbers of risk alleles; and 2) a greater increase in mean aneurysm expansion in women than men corresponds to the numbers of risk alleles.

Associations of age, baseline aneurysm size, PP and DAB21IP-rs7025486[A] with aneurysm expansion were also different in women and men. For example, an older age, higher PP, and a greater baseline aneurysm size had greater impact in women than men on aneurysm expansion (Table 17). Interactions of gender*0, 1 and 2 A alleles of rs7025486 with aneurysm expansion are shown in FIG. 6.

Multivariable stepwise regression analysis identified baseline aneurysm size, MAP, PP, PP*gender, DAB2IP-rs7025486[A], DAB2IP-rs7025486[A] *gender SORT1 to be independently associated with aneurysm expansion (Table 18). While the association of DAB2IP-rs7025486[A] (per risk allele) with aneurysm expansion was stronger in women than men; higher PP was associated with greater aneurysm expansion in women.

The mean growth rate in this study population was 0.44 mm/year greater in women than men, per A allele of DAB2IP-rs7025486, and 0.30 mm/year greater in women than men for each 10 mm Hg increase in PP, after adjustment for MAP, baseline size and SORT1. With these gender modified associations of PP and DAB2IP-rs7025486[A] with aneurysm expansion in the same model, we assessed whether PP modified the association of gender*DAB2IP-rs7025486[A] with aneurysm expansion by including a three-way interaction term of PP*gender*DAB2IP-rs7025486[A]. The interaction was significantly associated with greater aneurysm expansion independent of MAP and baseline size (regression coefficient β=0.034, p=0.007, Table 17). Women had a mean growth rate 0.68 mm/year greater than men per A allele of rs7025486 in high PP (≧median) group, but not in low PP group (FIG. 7).

In additional analyses, we found that SBP, DBP and MAP, but not PP decreased over time, (FIG. 8). Change in BP did not modify the association of DAB2IP with aneurysm expansion. When baseline PP and MAP was used in the analysis, results were similar (Table 20).

Thus, a stronger association of DAB2IP-rs7025486[A] with aneurysm expansion in women in the setting of elevated PP is shown. Higher PP increases shear stress and aortic wall stress, thereby increasing risk for aneurysm expansion (Li, et al., “Association between Aneurysm Shoulder Stress and Abdominal Aortic Aneurysm Expansion: A Longitudinal Follow-up Study.” Circulation, 122:1815-1822 2010). Thus, in one embodiment, women and men with at least one DAB2IP-rs7025486[A] should be considered at risk for aneurysm rupture and candidates for treatment to reduce the potential for aneurysm rupture. In another embodiment, women with two DAB2IP-rs7025486[A] alleles should be considered having a high risk of aneurysm rupture and candidates for treatment to reduce the potential for an impending aneurysm rupture.

As described herein, contemplated clinical implications of a genetic risk score (GRS) for rapid AAA expansion and the genetic basis for different AAA growth patterns was evaluated. Parameters evaluated: 1) whether a GRS for rapid AAA expansion consisting of variants with a p value <10E-5 in GWAS can predict faster aneurysm expansion; 2) whether incorporating genetic variants into clinical risk factors improves risk reclassification for rapid aneurysm expansion and rupture; 3) whether AAA expansion rates differ by growth pattern; and 4) whether functional variants from candidate gene analyses are also associated with faster aneurysm expansion and differ by AAA growth trajectory, using a mixed-effect model. These results are described in the Examples.

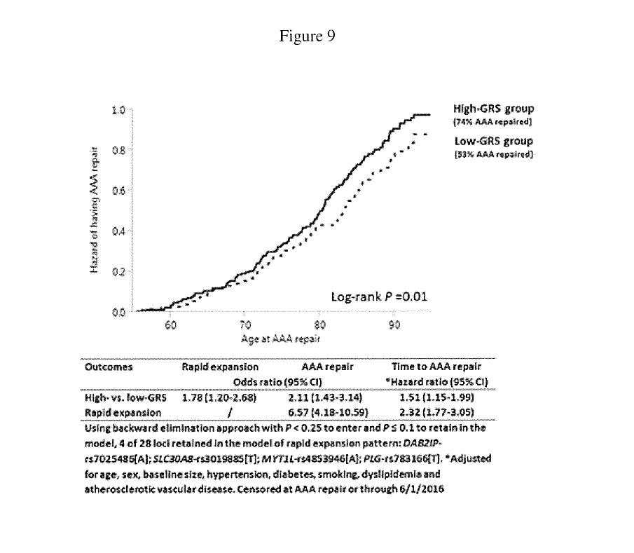

The following summarizes these results. A rapid expansion trajectory and high-GRS group were each associated with increased risk of aneurysm repair at younger age. See, FIG. 9.

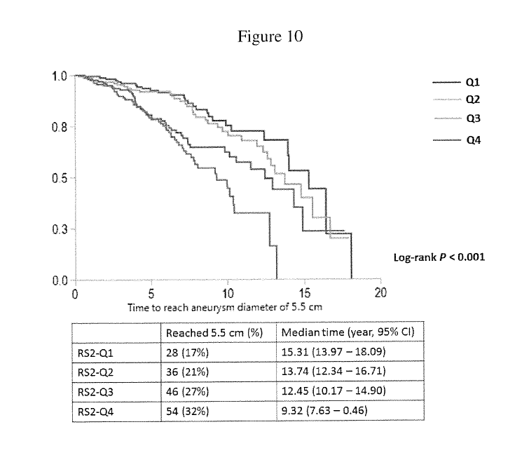

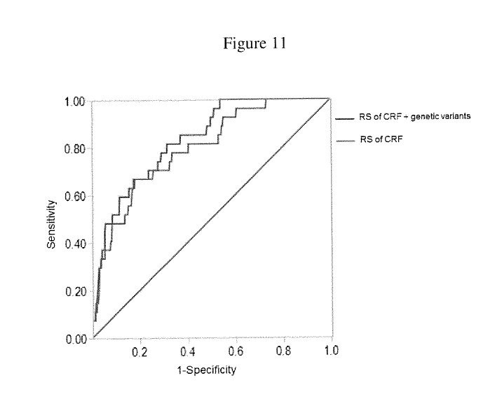

Compared with the model of baseline size alone, baseline size+RS1 (conventional risk factors only) did not improve disease discrimination or risk reclassification; while baseline size+RS2 (genetic variants+conventional risk factors) led to 17% improvement in risk reclassification (p for NRI=0.02, Table 24). Time to reach 5.5 cm stratified by quartiles of RS2 is shown in FIG. 10 using Kaplan-Meier analysis (log-rank p<0.001). See, FIG. 10. This chart demonstrates a Kaplan-Meier curve of quartiles of RS2: risk for expansion to a diameter of 5.5 cm.

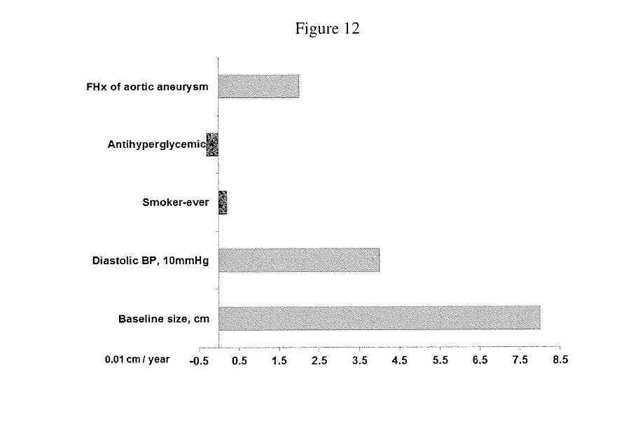

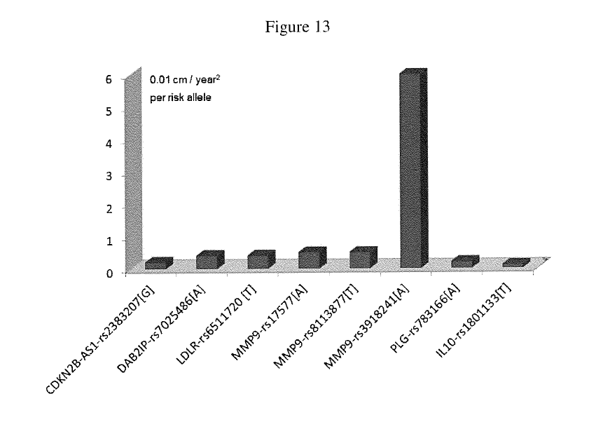

Adding genetic variants to clinical risk factors for rapid aneurysm expansion improved disease discrimination for AAA progression beyond clinical risk factors alone: such a risk score (RS) is associated with increased risk for AAA progressing to 5.5 cm and AAA rupture, leading to improved risk reclassification over a RS of clinical risk factors alone. See, FIG. 11. This chart demonstrates a C-statistic increase by genetic variants over clinical risk factors alone. See, FIG. 12. This chart demonstrates examples of clinical risk factors associated with faster AAA expansion.

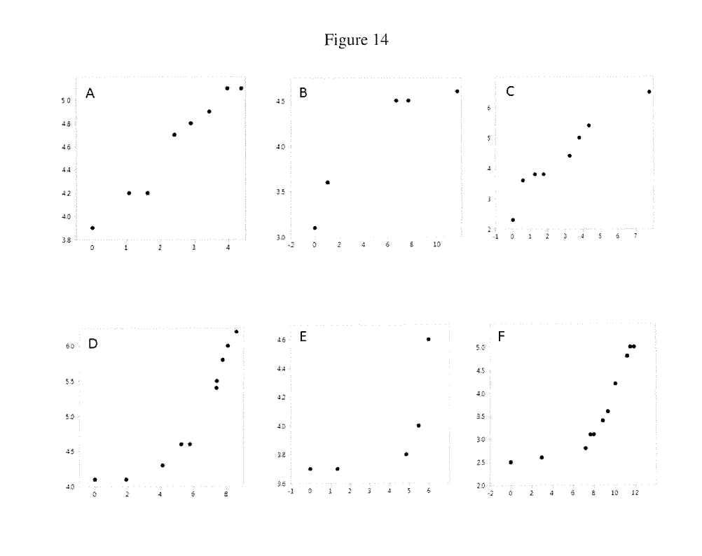

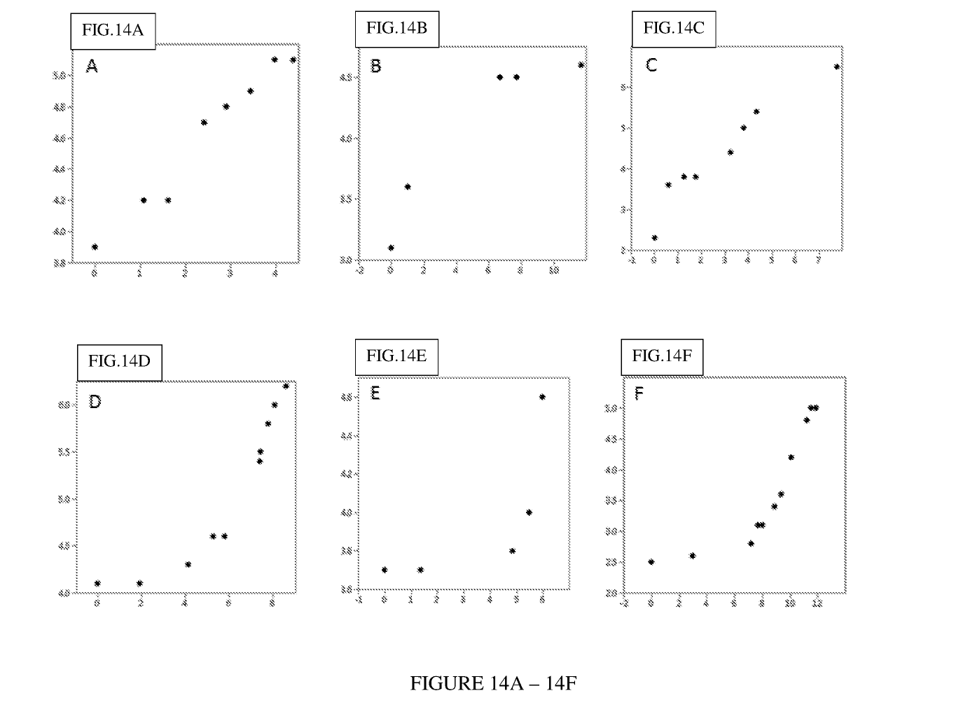

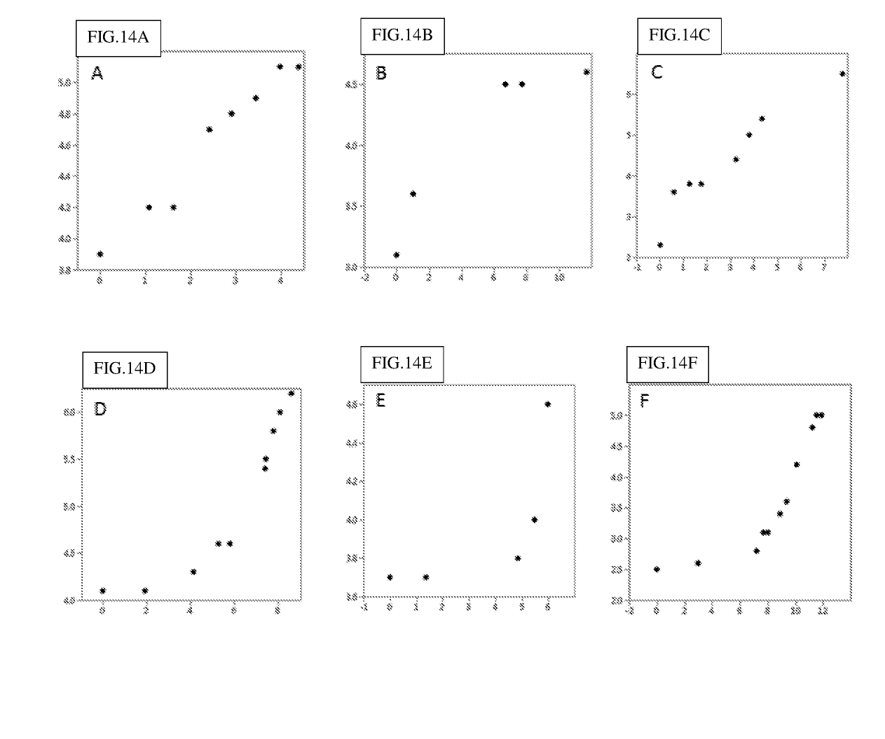

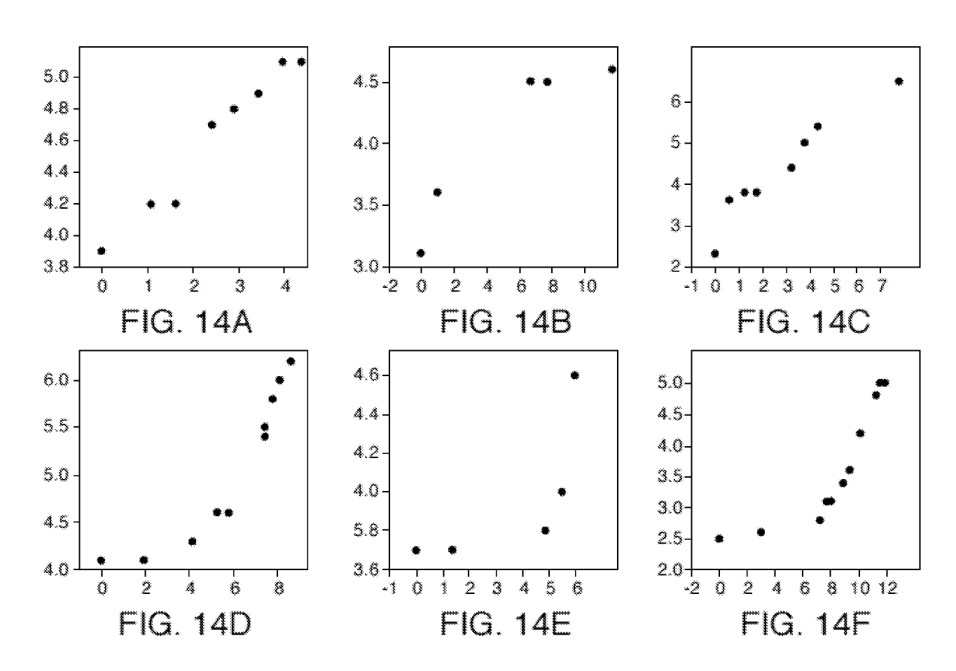

We identified two distinct growth patterns of AAA with different aneurysm behavior. EA was associated with increased risk of re-intervention and faster expansion than LA pattern. Genetic predisposition to AAA contributes to AAA expansion and susceptibility loci differently associated with growth pattern. See, FIG. 14. This chart demonstrates examples of an AAA expansion pattern: A-C: early-accelerated pattern; D-F: late-accelerated pattern.

### F. REFERENCES

Bhak, et al., “Factors Associated with Small Abdominal Aortic Aneurysm Expansion Rate.” JAMA Surg, 150(1):44-50 (2015).
Bown, et al., “Abdominal Aortic Aneurysm Is Associated with a Variant in Low-Density Lipoprotein Receptor-Related Protein 1.” Am J Hum Genet, 89(5):619-627 (2011 a).
Bown, et al., “Abdominal Aortic Aneurysm Is Associated with a Variant in Low-Density Lipoprotein Receptor-Related Protein 1.” Am J Hum Genet, 89(5):619-627 (2011 b).
Bown, et al., “Surveillance Intervals for Small Abdominal Aortic Aneurysms: A Meta-Analysis.” JAMA, 309(8):806-813 (2013).
Bradley, et al., “A Variant in Ldlr Is Associated with Abdominal Aortic Aneurysm.” CIrc Cardiovasc Genet, 6(5):498-504 (2013).
De Rango, et al., “Diabetes and Abdominal Aortic Aneurysms.” Eur J Vasc Endovasc Surg, 47(3):243-261 (2014).
Delaneau, et al., “A Linear Complexity Phasing Method for Thousands of Genomes.” Nat Methods, 9(2):179-181 (2012).
Devaraj and Dodds “Ultrasound Surveillance of Ectatic Abdominal Aortas.” Ann R Coll Surg Engl, 90(6):477-482 (2008).
Dichgans, et al., “Shared Genetic Susceptibility to Ischemic Stroke and Coronary Artery Disease: A Genome-Wide Analysis of Common Variants.” Stroke, 45(1):24-36 (2014).
Ding, et al., “Genotype-Informed Estimation of Risk of Coronary Heart Disease Based on Genome-Wide Association Data Linked to the Electronic Medical Record.” BMC Cardiovasc Disord, 11:66 (2011).
Ding, et al., “Gper-Independent Effects of Estrogen in Rat Aortic Vascular Endothelial Cells.” Mol Cell Endocrinol, 399:60-68 (2015).
Dua, et al., “Epidemiology of Aortic Aneurysm Repair in the United States from 2000 to 2010.” J Vasc Surg, 59(6):1512-1517 (2014).
Dube, et al., “Sortilin: An Unusual Suspect in Cholesterol Metabolism: From Gwas Identification to in Vivo Biochemical Analyses, Sortilin Has Been Identified as a Novel Mediator of Human Lipoprotein Metabolism.” Bioessays, 33(6):430-437 (2011).
Duellman, et al., “Analysis of Multiple Genetic Polymorphisms in Aggressive-Growing and Slow-Growing Abdominal Aortic Aneurysms.” J Vasc Surg, 60(3):613-621 e613 (2014).
Duggan, et al., “Two Genome-Wide Association Studies of Aggressive Prostate Cancer Implicate Putative Prostate Tumor Suppressor Gene Dab2ip.” J Natl Cancer Inst, 99(24):1836-1844 (2007).
Eric K. Shang. (2014). Increased Peak Wall Stress in Women with Abdominal Aortic Aneurysms. Paper presented at the Society for Clinical Vascular Surgery 42nd Annual symposium, La Costa Hotel, Carlsbad, Calif.
Freiberg, et al., “Abdominal Aortic Aneurysms, Increasing Infrarenal Aortic Diameter, and Risk of Total Mortality and Incident Cardiovascular Disease Events: 10-Year Follow-up Data from the Cardiovascular Health Study.” Circulation, 117(8):1010-1017 (2008).
Gretarsdottir, et al., “Genome-Wide Association Study Identifies a Sequence Variant within the Dab2ip Gene Conferring Susceptibility to Abdominal Aortic Aneurysm.” Nat Genet, 42(8):692-697 (2010).
Gretarsdottir, et al., “Genome-Wide Association Study Identifies a Sequence Variant within the Dab2ip Gene Conferring Susceptibility to Abdominal Aortic Aneurysm.” Nature genetics, 42(8):692-697 (2010).
Guirguis-Blake, et al., “Primary Care Screening for Abdominal Aortic Aneurysm: A Systematic Evidence Review for the U.S. Preventive Services Task Force.” Agency for Healthcare Research and Quality (US) (2014).
Harrison, et al., “Association of a Sequence Variant in Dab2ip with Coronary Heart Disease.” Eur Heart J, 33(7):881-888 (2012).
Helgadottir, et al., “The Same Sequence Variant on 9p21 Associates with Myocardial Infarction, Abdominal Aortic Aneurysm and Intracranial Aneurysm.” Nature genetics, 40(2):217-224 (2008).
Howie, et al., “A Flexible and Accurate Genotype Imputation Method for the Next Generation of Genome-Wide Association Studies.” PLoS Genet, 5(6):e1000529 (2009).
Ji, et al., “Both Internalization and Aip1 Association Are Required for Tumor Necrosis Factor Receptor 2-Mediated Jnk Signaling.” Arterioscler Thromb Vasc Biol, 32(9):2271-2279 (2012).
Jones, et al., “A Sequence Variant Associated with Sortilin-1 (Sort1) on 1p13.3 Is Independently Associated with Abdominal Aortic Aneurysm.” Hum Mol Genet, 22(14):2941-2947 (2013).
Kathiresan, et al., “Common Variants at 30 Loci Contribute to Polygenic Dyslipidemia.” Nature genetics, 41(1):56-65 (2009).
Kullo, et al., “Leveraging Informatics for Genetic Studies: Use of the Electronic Medical Record to Enable a Genome-Wide Association Study of Peripheral Arterial Disease.” J Am Med Inform Assoc, 17(5):568-574 (2010).
Laser, et al., “Increased Estrogen Receptor Alpha in Experimental Aortic Aneurysms in Females Compared with Males.” J Surg Res, 186(1):467-474 (2014).
Lemarie, et al., “Extracellular Matrix Alterations in Hypertensive Vascular Remodeling.” J Mol Cell Cardol, 48(3):433-439 (2010).
Lettre, et al., “Genome-Wide Association Study of Coronary Heart Disease and Its Risk Factors in 8,090 African Americans: The Nhlbi Care Project.” PLoS Genet, 7(2):e1001300 (2011).
Li, et al., “Association between Aneurysm Shoulder Stress and Abdominal Aortic Aneurysm Expansion: A Longitudinal Follow-up Study.” Circulation, 122(18):1815-1822 (2010).
Linsel-Nitschke, et al., “Genetic Variation at Chromosome 1p13.3 Affects Sortilin Mrna Expression, Cellular Ldl-Uptake and Serum Ldl Levels Which Translates to the Risk of Coronary Artery Disease.” Atherosclerosis, 208(1):183-189 (2010).
Lu, et al., “Dietary Phytoestrogens Inhibit Experimental Aneurysm Formation in Male Mice.” J Surg Res, 188(1):326-338 (2014).
Makrygiannis, et al., “Sex Differences in Abdominal Aortic Aneurysm: The Role of Sex Hormones.” Ann Vasc Surg 28(8):1946-1958 (2014).
Mitchell “Arterial Stiffness and Wave Reflection in Hypertension: Pathophysiologic and Therapeutic Implications.” Curr Hypertens Rep, 6(6):436-441 (2004).
Mitchell, et al., “Hemodynamics of Increased Pulse Pressure in Older Women in the Community-Based Age, Gene/Environment Susceptibility-Reykjavik Study.” Hypertension, 51(4): 1123-1128 (2008).
Mortensen, et al., “Targeting Sortilin in Immune Cells Reduces Proinflammatory Cytokines and Atherosclerosis.” J Clin Invest, 124(12):5317-5322 (2014).
Musunuru, et al., “From Noncoding Variant to Phenotype Via Sort1 at the lpl3 Cholesterol Locus.” Nature, 466(7307):714-719 (2010).
Nordon, et al., “Pathophysiology and Epidemiology of Abdominal Aortic Aneurysms.” Nat Rev Cardiol, 8(2):92-102 (2011).
Norman and Powell “Abdominal Aortic Aneurysm: The Prognosis in Women Is Worse Than in Men.” Circulation, 115(22):2865-2869 (2007).
Patel. et al., “Macrophage Sortilin Promotes Ldl Uptake, Foam Cell Formation, and Atherosclerosis.” Circ Res, 116(5):789-796 (2015).
Raaz, et al., “Segmental Aortic Stiffening Contributes to Experimental Abdominal Aortic Aneurysm Development.” Circulation, 131(20):1783-1795 (2015).
Reilly, et al., “Identification of Adamts7 as a Novel Locus for Coronary Atherosclerosis and Association of Abo with Myocardial Infarction in the Presence of Coronary Atherosclerosis: Two Genome-Wide Association Studies.” Lancet, 377(9763):383-392 (2011).
Sandhu, et al., “Ldl-Cholesterol Concentrations: A Genome-Wide Association Study.” Lancet, 371(9611):483-491 (2008).
Saratzis and Bown “The Genetic Basis for Aortic Aneurysmal Disease.” Heart, 100(12):916-922 (2014).
Schmit, et al., “Hypertension Overrides the Protective Effect of Female Hormones on the Development of Aortic Aneurysm Secondary to Alk5 Deficiency Via Erk Activation.” Am J Physiol Heart Circ Physiol, 308(2):H115-125 (2015).
Schunkert, et al., “Large-Scale Association Analysis Identifies 13 New Susceptibility Loci for Coronary Artery Disease.” Nature genetics, 43(4):333-338 (2011).
Strong, et al., “Hepatic Sortilin Regulates Both Apolipoprotein B Secretion and Ldl Catabolism.” J Clin invest, 122(8):2807-2816 (2012).
Sweeting, et al., “Meta-Analysis of Individual Patient Data to Examine Factors Affecting Growth and Rupture of Small Abdominal Aortic Aneurysms.” Br J Surg, 99(5):655-665 (2012).
Teslovich, et al., “Biological, Clinical and Population Relevance of 95 Loci for Blood Lipids.” Nature, 466(7307):707-713 (2010).
Wallace, et al., “Genome-Wide Association Study Identifies Genes for Biomarkers of Cardiovascular Disease: Serum Urate and Dyslipidemia.” Am J Hum Genet, 82(1):139-149 (2008).
Willer, et al., “Newly Identified Loci That Influence Lipid Concentrations and Risk of Coronary Artery Disease.” Nature genetics, 40(2):161-169 (2008).
Xie, et al., “Role of Dab2ip in Modulating Epithelial-to-Mesenchymal Transition and Prostate Cancer Metastasis.” Proc Natl Acad Sci USA, 107(6):2485-2490 (2010).
Ye. et al., “An Electronic Medical Record-Linked Biorepository to Identify Novel Biomarkers for Atherosclerotic Cardiovascular Disease.” Glob Cardiol Sci Pract, 2013(1):82-90 (2013).
Ye, et al, “Abstract 37: A Multi-Locus Genetic Risk Score for Abdominal Aortic Aneurysm.” Arteriosclerosis, Thrombosis, and Vascular Biology, 35(Suppl 1):A37 (2015).
Yeh, et al., “Infiltrating T Cells Promote Renal Cell Carcinoma (Rcc) Progression Via Altering the Estrogen Receptor Beta-Dab2ip Signals.” Oncotarget (2015).
Yu, et al., “Aip1 Prevents Graft Arteriosclerosis by Inhibiting Interferon-Gamma-Dependent Smooth Muscle Cell Proliferation and Intimal Expansion.” Circ Res, 109(4):418-427 (2011).
Zhang, et al., “Aip1-Mediated Stress Signaling in Atherosclerosis and Arteriosclerosis.” Curr Atheroscler Rep, 17(5):503 (2015).
Zhou, et al., “Aip1 Mediates Vascular Endothelial Cell Growth Factor Receptor-3-Dependent Angiogenic and Lymphangiogenic Responses.” Arterioscler Thromb Vase Biol, 34(3):603-615 (2014).

The NHGRI-EBI Catalog of published genome-wide association studies is available at: www.ebi.ac.uk/gwas. Last data release on 2016-04-24, Genome assembly GRCh38.p5, dbSNP Build 146, Ensembl Build 84. NHGRI-EBI GWAS Catalog data is cited to Burdett T (EBI), Hall P N (NHGRI), Hastings E (EBI), Hindorff L A (NHGRI), Junkins H A (NHGRI), Klemm A K (NHGRI), MacArthur J (EBI), Manolio T A (NHGRI), Morales J (EBI), Parkinson H (EBI) and Welter D (EBI). Publications related to The NHGRI-EBI GWAS Catalog include: Welter, et al., “The NHGRI GWAS Catalog, a curated resource of SNP-trait associations.” Nucleic Acids Research, 2014, Vol. 42 (Database issue): D1001-D1006; Hindorff, et al., “Potential etiologic and functional implications of genome-wide association loci for human diseases and traits.” Proc. Natl Acad. Sci. USA, 2009, 106, 9362-9367.

### EXPERIMENTAL

The following examples serve to illustrate certain embodiments and aspects of the present invention and are not to be construed as limiting the scope thereof.

The following abbreviations apply: AAA, abdominal aortic aneurysm; ASCVD, atherosclerotic cardiovascular disease; CHD, coronary heart disease; CI, confidence interval; EHR, electronic health record; GWAS, Genome-wide association studies; OR, odds ratio; SNP, Single nucleotide polymorphism; T2D, Type 2 diabetes, and sex=gender.

### Example I

The following is an exemplary calculation of the genetic risk score (GRS) as a rescaled weighted genetic risk score (r_GRS_W) for use as a GRS as described herein. In one embodiment, a GRS may be calculated for providing a GRS for a population of individuals. In another embodiment, a GRS is used for calculating a score for an individual patient. Thus

$$r\_GRS \_W = \frac{k}{{\sum }_{i}^{ } {w}_{i}} {\sum }_{i}^{ } {w}_{i} \times {n}_{i} ,$$

is r_GRS_W=k/Σᵢwᵢ Σ/i wᵢ×ηᵢ.

The weighted score equation was derived based on the assumption that the SNPs of interest have independent effects on the disease and contribute to the log risk of the disease in an additive manner. Lin, et al., 2009. The rescaled version of the genetic score shown above, uses a rescaling factor in order to provide a weighted genetic score more comparable to the unweighted genetic score for a cumulative number of alleles. Lin, et al., 2009. An example of steps to construct the parts of this equation are as follows.

A patient is genotyped, from a blood sample or a tissue sample, for having a particular risk allele SNP. Then each SNP is assigned a code, i.e. ‘0’ for a non-risk allele homozygote, ‘1’ for a risk-allele SNP heterozygote, and ‘2’ for both alleles having the risk-allele SNP, i.e. a risk-allele homozygote. Thus SNPᵢ=0, 1 or 2 according to the number of risk alleles for the specific locus in an individual. When a population is used for providing a genetic risk score, then the SNPᵢ is a sum of the codes for each allele for the entire population. In an example where SNP₁=rs7025486(A), SNPᵢ has a value of 2 for a patient having 2 risk alleles for rs7025486(A), etc. When there are 3 individuals in a population, one a non-risk allele homozygote, one a risk-allele SNP heterozygote and one a risk-allele homozygote, then SNP_{rs7025486(A)}=0+1+2=3 for use in the equation. ηᵢ is the number of risk alleles for SNPᵢ, for example, when 4 risk alleles are used, then i=1, 2, 3, and 4, with each of the 4 alleles assigned a separate number.

When combining multiple SNPs, a weighted genetic score calculation is used based upon a weighted w value calculated for each allele, i.e. wᵢ, for SNPᵢ. Thus, wᵢ=the logarithm of odds ratio (OR at a 95% CI), calculated for each allele based upon that allele's estimated effect size obtained from a GWAS catalog or published largest meta-analysis. For examples of an OR for each allele, see Table 1 showing OR values obtained from the GWAS catalog at NHGRI-EBI Catalog of published genome-wide association studies https://www.ebi.ac.uk/gwas/search?query=ABDOMINAL AORTIC ANEURYSM#association. Thus, wᵢ=log(ORᵢ). So for a weighted genetic risk score, with allele counts across several SNPs, weighted by the logarithm of odds ratio=w₁×SNP₁+w₂×SNP₂+ . . . wᵢ×SNPᵢ.

Then a rescaling factor is used=k/Σᵢwᵢ, where k is the number of SNPs used (i.e. k=4 for a 4 SNP allele calculation), for a rescaled weighted genetic score, calculated by summing k×(w₁×SNP₁+w₂×SNP₂+ . . . wᵢ×SNPᵢ)/(w₁+w₂+ . . . wᵢ).

Equations and calculations are generally described in: (K. Ding, et al., “Genotype-Informed Estimation of Risk of Coronary Heart Disease Based on Genome-Wide Association Data Linked to the Electronic Medical Record.” BMC Cardiovasc Disord, 11:66 2011); (Lin, et al, “Risk prediction of prevalent diabetes in a Swiss population using a weighted genetic score-the CoLaus Study.” Diabetologia, 52(4):600-608, 2009).

A median, i.e. middle, is determined as the middle number of the numbers when lined up lowest to highest. When there are two middle numbers instead of one, then determine the value half way in between these two numbers, i.e. add the two middle numbers together then divide by two.

### Example II

The following is an exemplary use of a median related to identifying individual patients with AAA using a GRS medium.

The study comprised of 1098 patients with AAA (74±8 years, 83% men) and 6538 controls (67±10 years, 58% men) enrolled in the Mayo Vascular Disease Biorepository. AAA was defined as a transverse diameter of abdominal aorta ≧3.0 cm or history of AAA repair. Controls were participants without known AAA. A GRS for AAA for each individual was calculated from four SNPs (rs2383107, rs7025486, rs599839, rs1466535) that were replicated in our cohort/population, by summing the number of risk alleles for each SNP weighted by their estimated effect sizes in GWAS catalog or published largest meta-analysis.

GRS was associated with presence of AAA: odds ratio (OR) (95% confidence interval): 1.06 (1.03-1.08). The association remained significant after adjustment for age, sex, cardiovascular risk factors, and atherosclerotic cardiovascular diseases: adjusted OR: 1.05 (1.03-1.08). In this example, adjustment for each SNP did not attenuate association of GRS with presence of AAA (each SNP P<0.001). GRS was not associated with family history of aortic aneurysm (P=4). Adding GRS to conventional risk factors improved net reclassification index by 16% (P<0.001).

In a subset of patients with AAA who had sequential imaging studies (n=628), GRS was associated with AAA growth rate ≧1.75 mm/year (median of the cohort) after adjustment for baseline AAA size: adjusted OR: 1.07 (1.00-1.14). No conventional risk factors were associated with AAA growth.

Patients with GRS>5.24 (median of the cohort) had 1.31 times higher odds of having AAA (P≦0.005) and 1.64 times higher odds of having AAA growth rate ≧1.75 mm/year compared to those with GRS≦5.24 (P50.005).

### Example III

The following is an exemplary determination of the probability of aneurysm expansion when at least one allele for rs7025486[A] is present in men and women.

Of 5 genetic susceptibility variants for AAA (Table 17), DAB2IP-rs7025486[A] and SORT1-rs599839[G] were associated with aneurysm expansion. However, the mean aneurysm expansion was 0.5 mm/year greater per A allele of DAB2IP-rs7025486 (p<0.01) over 0.44 mm/year greater per G allele of SORT1-rs599839 (p<0.01). While association of SORT1-rs599839[G] was similar in women and men, in women DAB2IP-rs7025486[A] showed a mean growth rate 0.47 mm/year greater than men per A allele of DAB2IP-rs7025486 (p=0.02). FIG. 6 illustrates: 1) an increase in mean aneurysm expansion that corresponds to the numbers of risk alleles; and 2) a greater increase in mean aneurysm expansion in women than men corresponds to the numbers of risk alleles.

Associations of age, baseline aneurysm size, PP and DAB2IP-rs7025486[A] with aneurysm expansion were also different in women and men. For example, an older age, higher PP, and a greater baseline aneurysm size had greater impact in women than men on aneurysm expansion (Table 17). Interactions of gender*0, 1 and 2 A alleles of rs7025486 with aneurysm expansion are shown in FIG. 6.

Multivariable stepwise regression analysis identified baseline aneurysm size, MAP, PP, PP*gender, DAB2IP-rs7025486[A], DAB2IP-rs7025486[A]*gender, SORT to be independently associated with aneurysm expansion (Table 18). While the association of DAB2IP-rs7025486[A] (per risk allele) with aneurysm expansion was stronger in women than men; higher PP was associated with greater aneurysm expansion in women.

The mean growth rate in this study population was 0.44 mm/year greater in women than men, per A allele of DAB2IP-rs7025486, and 0.30 mm/year greater in women than men for each 10 mm Hg increase in PP, after adjustment for MAP, baseline size and SORT1. With these gender modified associations of PP and DAB2IP-rs7025486[A] with aneurysm expansion in the same model, we assessed whether PP modified the association of gender*DAB2IP-rs7025486[A] with aneurysm expansion by including a three-way interaction term of PP*gender* DAB2IP-rs7025486[A]. The interaction was significantly associated with greater aneurysm expansion independent of MAP and baseline size (regression coefficient β=0.034, p=0.007, Table 17). Women had a mean growth rate 0.68 mm/year greater than men per A allele of rs7025486 in high PP (≧median) group, but not in low PP group (FIG. 7).

In additional analyses, we found that SBP, DBP and MAP, but not PP decreased over time, (FIG. 8). Change in BP did not modify the association of DAB2IP with aneurysm expansion. When baseline PP and MAP was used in the analysis, results were similar (Table 20).

Thus, a stronger association of DAB2IP-rs7025486[A] with aneurysm expansion in women in the setting of elevated PP is shown. Higher PP increases shear stress and aortic wall stress, thereby increasing risk for aneurysm expansion (Li, et al., “Association between Aneurysm Shoulder Stress and Abdominal Aortic Aneurysm Expansion: A Longitudinal Follow-up Study.” Circulation, 122:1815-1822 2010).

Thus, in one embodiment, women and men with at least one DAB2IP-rs7025486[A] should be considered at risk for aneurysm rupture and candidates for treatment to reduce the potential for aneurysm rupture. In another embodiment, women with two DAB2IP-rs7025486[A] alleles should be considered having a high risk of aneurysm rupture and candidates for treatment to reduce the potential for an impending aneurysm rupture.

### Example IV

The following is an example for determining a genetic risk score (GRS) for predicting rapid AAA expansion.

We identified 427 patients (84% men) from the Mayo clinic vascular disease biorepository who had AAA and with ≧2 measures of pre-operative AAA size assessed at least 3 months apart (all by abdominal computed tomography). A mixed-effect model was used to estimate AAA expansion over time. Random effects were included to account for variation in observational time among individuals that allowed individual deviation from the mean growth rate of the cohort. Baseline size was added as a covariate to provide a better fit of the model. We defined rapid AAA expansion as an individual expansion rate ≧95% confidence interval of the mean expansion rate of the cohort. NHGRIEBI GWAS Catalog and PubMed were used to search genetic susceptibility variants for AAA at p<10×10̂5.

A weighted GRS using effect sizes from the catalog or largest metaanalysis was constructed comprising of genetic variants associated with rapid AAA expansion trajectory individual AAA expansion over time>upper limit of 95% confidence interval (CI) of mean AAA expansion rate of the cohort (4 of 28 loci identified by stepwise elimination approach).

Results: The mean baseline AAA size of the cohort was 4.0±0.85 cm with a mean follow up of 4.1≡3.3 years. After adjustment for baseline size, mean AAA expansion was 0.21 (95% CI: 0.19 0.23) cm/year, 156 patients had rapid AAA expansion trajectory [mean (SE) AAA expansion 0.3 (0.01) cm/year]. Age, sex, prevalence of cardiovascular risk factors and atherosclerotic vascular diseases were similar in high (>median) and lowGRS groups. None were associated with rapid expansion trajectory (all P>0.05). HighGRS group was more likely to have rapid expansion and aneurysm repair than low GRS group.

Rapid expansion trajectory and highGRS group were each associated with increased risk of aneurysm repair at younger age. See, FIG. 9. This chart demonstrates a rapid expansion trajectory and a highGRS group where each associated with increased risk of aneurysm repair at a younger age.

### Example V

The following is an example showing risk reclassification for rapid AAA expansion and aneurysm rupture by genetic variants over conventional risk factors.

Adding genetic variants to clinical risk factors for rapid aneurysm expansion improved disease discrimination for AAA progression beyond clinical risk factors alone: such a risk score (RS) is associated with increased risk for AAA progressing to 5.5 cm and AAA rupture, leading to improved risk reclassification over a RS of clinical risk factors alone.

A. AAA Growth.

Four thousand two hundred twenty one (4221) measures of sequential AAA sizes for each patient were abstracted from radiology reports. Data element included: date of the imaging study, imaging modality, maximal AP diameter from ultrasound (US) or maximal cross-sectional diameter from CT; repair date; repair type. Small AAA is often followed by US. Not until AAA diameter reaches certain threshold will CT be initiated.

To observe the entire spectrum of AAA growth, we included measures from both US and CT. Observational time for each patient was from the first measure in the EHR until the last measure before Oct. 1, 2016 or before AAA repair. Of 4221 measures of pre-operative AAA size abstracted from the EHR, 2349 (56%) were measured by US and remaining by CT. We assessed differences in measures from US and CT in 196 pairs evaluated within one month.

We found that 36% of them with no difference and 56% with a difference <0.5 cm (Table 21). For the purpose of the current study, we included 708 patients with ≧2 measures of AAA size assessed ≦3 months apart. For measures assessed >3 months by different imaging modalities, a correction term for the size was used to keep the consistency in measures. Of 708 patients with 3644 measures in total, 216 patients (31%) had all sizes measured by CT and 166 patients (23%) had all sizes measured by US. We included patients without AAA who had ≧2 abdominal aortic imaging studies assessed ≧5 years (n=1692) as controls for AAA expansion.

TABLE 21
Differences IN AAA Size Measured By Ultrasound-CT
Within 30 Days (n = 196 pair).

| | AAA size | | | | | | |
|---|---|---|---|---|---|---|---|
| | 0 | 0 to | 0.1 to | 0.2 to | 0.3 to | 0.4 to | >0.5 |
| | cm | 0.1 cm | 0.2 cm | 0.3 cm | 0.4 cm | 0.5 cm | cm |
| No. | 70 | 36 | 25 | 17 | 20 | 13 | 6 |
| | (36%) | (18%) | (13%) | (8%) | (10%) | (7%) | (8%) |

We used generalized logistic regression analysis with forward stepwise approach to select most significant genetic variants and CRFs to build a risk score (RS) for rapid AAA expansion and a RS for slow AAA expansion based on AIC criteria. The RS was the sum of intercept and variables retained in the model weighted by the corresponding log-odds of the regression-coefficients.

To assess the clinical utility of the RS, we tested 1) whether RS for rapid AAA expansion could improve prediction for AAA expansion over baseline size, using time to reach 5.5 cm (the threshold for aneurysm repair) as a time-dependent variable by Cox proportional hazard analysis; and 2) whether the same RS can improve disease discrimination for AAA rupture over CRFs alone. C-statistic increase, net-reclassification index (NRI) and integrated discrimination index (IDI) were used to assess improvement in disease discrimination and risk reclassification by RS of genetic variants+CRFs over RS of CRFs alone. See, FIG. 11. This chart demonstrates a C-statistic increase by genetic variants over clinical risk factors alone.

B. Patient Characteristics Stratified by Expansion Group.

Characteristics of controls and patients with rapid or slow expansion are shown in Table 22. Briefly, patients with AAA were older and had more clinical risk factors than controls. Compared with patients with slow expansion, patients with rapid expansion had higher DBP, higher glucose level, and were less likely to have dyslipidemia (Table 23). Mean growth rate were 0.31 (0.30 to 0.31) cm/year and 0.12 (0.11 to 0.13) cm/year in two expansion groups respectively. Patients with rapid expansion had larger baseline size and shorter observational time (Table 22).

TABLE 22
Difference in AAA Behavior In Two Groups: Slow And Rapid Expansion Rates.

| | In all | Slow expansion | Rapid expansion |
|---|---|---|---|
| | N = 708 | (n = 432) | (n = 276) |
| Baseline size, cm | 3.59 | 3.40 | 3.90 |
| | (3.65 to 3.54) | (3.33 to 3.47) | (3.81 to 3.99) |
| Last or pre-operative size, | 4.65 | 4.20 | 5.35 |
| cm | (4.57 to 4.73) | (4.12 to 4.29) | (5.24 to 5.46) |
| No of measures of AAA | 5.18 | 5.48 | 4.73 |
| size | (4.96 to 5.42) | (52.19 to 5.77) | (4.37 to 5.10) |
| Observational time, year | 5.89 | 7.00 | 4.16 |
| | (5.60 to 6.18) | (6.66 to 7.34) | (3.73 to 4.59) |
| Adjusted growth rate, cm/ | 0.19 | 0.12 | 0.31 |
| year | (0.18 to 0.20) | (0.11 to 0.13) | (0.30 to 0.31) |

TABLE 23
Exemplary Patient Characteristics.

| | Controls | Slow expansion | Rapid expansion |
|---|---|---|---|
| | (N = 1692) | (n = 432) | (n = 276) |
| ** Age, years | 64.22 (9.91) | 70.24 (7.37) | 69.21 (7.63) |
| ** Women, % | 41 | 16 | 17 |
| Body mass index, | 29.14 (5.35) | 29.30 (4.31) | 28.75 (452) |
| kg/m2 | | | |
| Systolic BP, mmHg | 131.65 (12.33) | 132.04 (11.30) | 131.83 (11.61) |
| *‡ Diastolic BP, | 72.58 (9.30) | 71.79 (6.35) | 73.43 (7.33) |
| mmHg | | | |
| ** Hypertension | 79 | 84 | 85 |
| Type 2 diabetes | 27 | 26 | 25 |
| ** Smoking (ever) | 64 | 90 | 84 |
| **† Dyslipidemia | 86 | 92 | 89 |
| ASCVD | 77 | 90 | 89 |
| ** COPD | 14 | 31 | 30 |
| ** FHx of aortic | 9 | 14 | 17 |
| aneurysm | | | |
| ** TC, mg/dL | 182.82 (30.43) | 173.94 (30.71) | 174.56 (32.03) |
| ** LDL, mg/dL | 100.26 (24.08) | 96.12 (25.19) | 98.01 (27.73) |
| ** HDL, mg/dL | 52.78 (15.08) | 47.33 (12.39) | 45.63 (12.52) |
| **‡ Glucose, | 108.20 (12.24) | 110.87 (12.01) | 113.34 (10.83) |
| mg/dL | | | |
| ** Statin use | 80 | 86 | 83 |
| Antihyperglycemic | 22 | 18 | 18 |
| use | | | |
| Antihypertensive | 82 | 86 | 82 |
| use | | | |

* p for trend < 0.05;
** p for trend < 0.01;
†p < 0.05 for slow vs. rapid expansion;
‡p < 0.05 for slow vs. rapid expansion;
ASCVD = atherosclerotic cardiovascular diseases;
COPD = chronic obstructive pulmonary disease;
FHx = family history;
TC = total cholesterol;
LDL = low density lipoprotein; and
HDL = high density lipoprotein.

C. Associations of RSs with Risk for Expansion to 5.5 cm and AAA Rupture.

Using generalized logistic regression analysis with forward stepwise selection in patients with rapid expansion and controls, two RSs were built: RS1 consisted of CRFs only, and RS2 of genetic variants and CRFs. The RS was highest in rapid expansion group and lowest in controls (p<0.01 from ANOVA). The C-statistics of RS1 and RS2 for rapid expansion were 0.82 and 0.84 respectively. There was significant improvement in disease discrimination for rapid AAA expansion by RS2 over RS1 (Δc-statistics=0.02, p<0.001). Bootstrapping with 1000 iterations demonstrated consistent results with a 95% CI of 0.80-0.84 for RS1 and of 0.81-0.86 for RS2.

Six hundred eighty five of 708 patients had a baseline AAA size <5 cm; and 167 of 685 reached an AAA diameter of 5.5 cm during the observational time. Cox PH models of baseline size alone, baseline size+RS1, and baseline size+RS2 are shown in Table 24. Baseline size, RS1 and RS2 were all associated with increased risk for faster expansion to reach 5.5 cm.

Compared with the model of baseline size alone, baseline size+RS1 (conventional risk factors only) did not improve disease discrimination or risk reclassification; while baseline size+RS2 (genetic variants+conventional risk factors) led to 17% improvement in risk reclassification (p for NRI=0.02, Table 24).

Time to reach 5.5 cm stratified by quartiles of RS2 is shown in FIG. 10 using Kaplan-Meier analysis (log-rank p<0.001). See, FIG. 10. This chart demonstrates a Kaplan-Meier curve of quartiles of RS2: risk for expansion to a diameter of 5.5 cm.

TABLE 24
Cox Proportional Hazard Model For Timing To Reach 5.5 cm

| | | Hazard | | | |
|---|---|---|---|---|---|
| | | ratio | 95% CI | NRI | IDI |
| Model 1 | Baseline | 8.99 | 6.87-11.84 | Ref | Ref |
| | size | | | | |
| Model 2 | Baseline | 8.34 | 6.37-11.01 | 0.11 (−0.03 | 0.14 (−0.08 |
| | size | | | to 0.32), | to 0.44), |
| | RS1 | 1.23 | 1.06-1.42 | p = 0.2 | p = 0.2 |
| Model 3 | Baseline | 8.31 | 6.36-10.93 | 0.17 (0.02 | 0.22 (−0.07 |
| | size | | | to 0.44), | to 0.75), |
| | RS2 | 1.28 | 1.12-1.46 | p = 0.02 | p = 0.08 |

RS1: clinical variables only;
RS2: clinical variables + genetic variants.
NRI and IDI were based on comparisons between model 2 vs. model 1; and model 3 vs. model 1.
Number of iterations for the perturbation-resampling = 500.

Of 1124 patients with AAA, 27 had ruptured AAA. The ORs and 95% CIs for AAA rupture of RS1 and RS2 were: 2.33, 1.77-3.10 and 2.32, 1.81-3.00 respectively, as compared with controls. AUC of RS2 for AAA rupture was higher than that of RS1 (FIG. 11). RS2 significantly improved risk reclassification for AAA rupture over RS1 (NRI=0.13, p=0.02). See, FIG. 11. This chart demonstrates a C-statistic increase by genetic variants over clinical risk factors alone. See, FIG. 12. This chart demonstrates examples of clinical risk factors associated with faster AAA expansion.

TABLE 25
Generalized Logistic Regression With Forward
Selection For Rapid AAA Expansion.

| | Rapid vs. controls | | |
|---|---|---|---|
| | β | SE | P-value |
| Intercept | −15.32 | 1.95 | <.01 |
| Age | 0.09 | 0.01 | <.01 |
| Women | −0.45 | 0.20 | 0.03 |
| COPD | 0.58 | 0.19 | <.01 |
| Smoking history | 0.93 | 0.20 | <.01 |
| Hypertension | 0.40 | 0.24 | 0.10 |
| Dyslipidemia | / | / | / |
| ASCVD | 0.73 | 0.23 | <.01 |
| T2D | −0.62 | 0.27 | 0.02 |
| HDL | −0.04 | 0.01 | <.01 |
| Glucose | 0.06 | 0.01 | <.01 |
| BMI | −0.05 | 0.02 | 0.01 |
| PP | −0.06 | 0.01 | <.01 |
| MAP | 0.08 | 0.02 | <.01 |
| FHx of aortic aneurysm | 0.91 | 0.23 | <.01 |
| Antihyperglycemic | −0.75 | 0.29 | 0.01 |
| Anti-hypertensive | −0.61 | 0.24 | 0.01 |
| CDKN2A-AS1 | 0.19 | 0.11 | 0.09 |
| rs10757278 | | | |
| DAB2IP-rs7025486 | 0.45 | 0.12 | <.01 |
| LRP1-rs1466535 | 0.20 | 0.12 | 0.08 |
| LHFPL2-rs1372319 | 0.24 | 0.13 | 0.07 |
| SLC15A5-rs1671518 | 0.25 | 0.12 | 0.04 |
| BMP4-rs2071047 | / | / | / |
| ERG-rs2836470 | 0.26 | 0.14 | 0.07 |
| GPC6-rs2892667 | 0.18 | 0.13 | 0.15 |
| MYT1L-rs4853946 | −0.28 | 0.11 | 0.01 |
| TDRD10-rs6674171 | 0.24 | 0.13 | 0.08 |
| LEP-rs6979784 | / | / | / |
| C9orf92-rs7044238 | / | / | / |
| TMEM247-rs7565770 | / | / | / |
| DYNC1I1-rs7798936 | / | / | / |
| KCNIP1-rs959461 | 0.34 | 0.14 | 0.02 |

### Example VI

The following shows an exemplary AAA expansion pattern with associated genetic risk factors.

We studied 486 patients, who had ≧3 pre-operation measures for AAA size and available high-density genotyping information in the Mayo clinic Vascular Disease Biorepository. We classified patients as having early (EA, n=268) vs. late-accelerated (LA, n=220) growth pattern according to individual growth curves. Clinical information was ascertained from electronic health records. Genetic variants for AAA were selected from genome-wide association study catalog with a p≦10E-5 and known functional variants from candidate gene studies.

AAA expansion was faster in EA than LA group (p<0.01). In patients who underwent AAA repair (n=234), the odds ratio of EA vs LA pattern for re-intervention was 2.75 (95% CI: 1.25-6.47) independent of potential confounders (Table 26). The associations of clinical variables with AAA expansion were not differed by the pattern (Table, p for interaction >0.05). Of 17 candidate genetic variants, 8 were associated with faster expansion and 3 associated with expansion differed by growth pattern (Table 26).

We identified two distinct growth patterns of AAA with different aneurysm behavior. EA was associated with increased risk of re-intervention and faster expansion than LA pattern. Genetic predisposition to AAA contributes to AAA expansion and susceptibility loci differently associated with growth pattern. See, FIG. 14A-F. This chart demonstrates examples of an AAA expansion pattern: FIG. 14A-C: early-accelerated pattern; FIG. 14D-F: late-accelerated pattern.

TABLE 26
Associations of clinical variables and genetic variants
with AAA expansion using linear mixed model.

| | Adjusted mean difference | Interact with |
|---|---|---|
| | (cm/year*; cm/year\hat{ }2†) | growth pattern |
| Age, 10 years | 0.001 (0.0001 to 0.002) † | No |
| Baseline size, cm | 0.08 (0.06 to 0.10) * | No |
| Diastolic BP, 10 mmHg | 0.04 (0.02 to 0.06) * | No |
| Smoking - ever | 0.002 (0.001 to 0.003) † | No |
| Antihyperglycemic | −0.003 (0.002 to 0.004) † | No |
| medication | | |
| Family history of aortic | 0.02 (0.001 to 0.03) * | No |
| aneurysm | | |
| CDKN2B-AS1- | 0.002 (0.001 to 0.003) † | No |
| rs2383207[G] | | |
| DAB2IP-rs7025486[A] | 0.004 (0.002 to 0.004) † | No |
| LRP1-rs1466351[C]‡ | 0 (−0.001 to 0.001) | p = 0.046 |
| LDLR-rs6511720 [T]‡ | 0.004 (0.002 to 0.006) † | p = 0.04 |
| MMP9-rs17577[A] | 0.005 (0.003 to 0.006) † | No |
| MMP9-rs8113877[T] | 0.004 (0.001 to 0.007) † | No |
| MMP9-rs3918241[A] | 0.06 (0.02 to 0.1) † | No |
| PLG-rs783166[A] | 0.002 (0 to 0.003) † | No |
| IL10-rs1801133[T] | 0.001 (0.0002 to 0.002) † | No |
| AGTR1-rs5186[C]‡ | 0.001 (−0.001 to 0.002) | p = 0.02 |

The mean age was 69 ± 7 years and baseline AAA size was 3.5 ± 0.7 cm, 83% were men.
Mean grow rate (95% CI) of EA (linear or logarithm growth curves) vs. LA (exponential or polynomial order ≧ 3) pattern: 0.22 (0.20 to 0.23) vs 0.14 (0.12 to 0.16) cm/year.
CDKN2B-AS1 = CDKN2B antisense RNA1;
DAB2IP = Disabled homolog 2-interacting protein;
LRP1 = low density lipoprotein receptor-related protein 1;
LDLR = low-density lipoprotein receptor;
MMP9 = matrix metallopeptidase 9;
IL10 = interleukin 10;
PLG = plasminogen;
AGTR1 = angiotensin-1 receptor.
‡Genetic variants associated with AAA expansion differed by pattern. LRP1-rs1466351[C] associated with faster expansion in LA group; LDLR-rs6511720[T] and AGTR1-rs5186[C] associated with faster expansion in EA group.
* or † with a p < 0.05;
Graft related complications that required re-intervention included endoleak, limb ischemia, postimplantation rupture or juxta-anastomotic aneurysmal information.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, molecular biology, cell biology, genetics, statistics or related fields are intended to be within the scope of the following claims.

1. A method for identifying and treating a high-risk aneurysm in an Abdominal Aortic Aneurysm (AAA) patient, comprising,
a) providing,
i) a sample of genomic DNA from an Abdominal Aortic Aneurysm (AAA) patient, and
ii) a weighted genetic risk score median calculated using a population of patients with AAA;
b) testing said DNA for a single nucleotide polymorphism (SNP) in each of four AAA risk alleles, wherein said risk alleles are rs1466535(C), rs7025486(A), rs2383207(T), and rs599839(G);
c) assigning a code for each said individual risk allele;
d) calculating a weighted genetic risk score for said patient using said codes for each allele;
e) determining that said weighted genetic risk score of said patient is greater than said median; and
f) treating said aneurysm of said AAA patient.

2. The method of claim 1, wherein said code is a 0 for a non-risk allele homozygote, a 1 for a heterozygote and a 2 for a risk allele heterozygote.

3. The method of claim 1, wherein said treating comprising surgical repair to prevent rupture of said aneurysm.

4. The method of claim 3, wherein a transverse diameter of said AAA is ≧3.0 cm before said surgical repair.

5. The method of claim 1, wherein said patient has history of AAA repair.

6. The method of claim 1, wherein said testing of step b) comprises sequencing at least a portion of said DNA sample.

7. The method of claim 1, wherein said weighted genetic risk score is a rescaled weighted genetic risk score.

8. A method for determining increased aneurysm expansion risk and treating an Abdominal Aortic Aneurysm (AAA) patient, comprising,
a) providing a sample of genomic DNA from an AAA patient;
b) testing said DNA for a single nucleotide polymorphism (SNP) in a single risk allele, where said risk allele is rs7025486; and
c) initiating a treatment when at least one SNP A is present in said allele rs7025486.

9. The method of claim 8, wherein said patient is a female.

10. The method of claim 8, wherein said testing of step b) comprises sequencing at least a portion of said DNA sample.

11. The method of claim 8, wherein said treatment is selected from the group consisting of an arterial de-stiffening and a surgical repair

12. The method of claim 11, wherein a second SNP A is present in said allele rs7025486 said treatment is surgical repair.

13. The method of claim 12, wherein a transverse diameter of said AAA is ≧3.0 cm before said surgical repair.

14. The method of claim 8, wherein said patient has history of AAA repair.
